(19) United States
(12) Patent Application Publication
(10) Pub. No.: US 2023/0374523 A1
(43) Pub. Date: Nov. 23, 2023

(54) CANCER THERAPY USING TOLL-LIKE RECEPTOR AGONISTS

(71) Applicant: TriSalus Life Sciences, Inc., Westminster, CO (US)

(72) Inventors: Steven C. Katz, East Greenwich, RI (US); Bryan F. Cox, Spring Grove, IL (US); David Benjamin Jaroch, Arvada, CO (US)

(73) Assignee: TriSalus Life Sciences, Inc., Westminster, CO (US)

(21) Appl. No.: 18/027,895

(22) PCT Filed: Sep. 21, 2021
(86) PCT No.: PCT/US2021/051384

§ 371 Date: Mar. 22, 2023

Related U.S. Application Data
(60) Provisional application No. 63/081,613, filed on Sep. 22, 2020.

(51) Int. Cl. C12N 15/117; A61K 39/395; A61K 39/39; A61P 35/00

(52) U.S. Cl. CPC C12N 15/117; A61K 39/39558; A61K 39/39; A61P 35/00; A61K 2039/54

(57) ABSTRACT

Embodiments of the present invention provide for methods of treating cancer and methods of delivering toll-like receptor (TLR) agonists to solid tumors in the pancreas using a locoregional therapy through the vasculature. In one aspect, the present invention relates to a method of treating pancreatic cancer comprising administering TLR agonists to the pancreas.

### CROSS-REFERENCE TO RELATED APPLICATION(S)

This application claims the benefit of U.S. Provisional Patent Application No. 63/081,613, which was filed on Sep. 22, 2020 and is incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 16, 2020, is named A372-502_SL.txt and is 484 bytes in size.

### FIELD OF THE INVENTION

The present disclosure relates generally to methods of treating cancer and methods of delivering toll-like receptor (TLR) agonists to solid tumors in the pancreas using a locoregional therapy through the vasculature.

### BACKGROUND OF THE INVENTION

Cancer is a devastating disease that involves the unchecked growth of cells, which may result in the growth of solid tumors in a variety of organs such as the skin, liver, and pancreas. Tumors may first present in any number of organs or may be the result of metastases or spread from other locations.

Pancreatic cancer is the third leading cause of cancer deaths in the United States, responsible for an estimated 55,000 deaths in 2018. The 5-year survival rate of this type of cancer is only 7-8%, which is attributed to various factors including the advanced stage of the disease at which the initial diagnosis often occurs, the propensity of this type of cancer to metastasize, the resistance of the disease to chemotherapy and radiation therapy, and the complex microenvironment of pancreatic cancer tumors. Only 15-20% of patients are eligible at diagnosis for surgical resection of the primary tumor, as most patients are initially diagnosed with unresectable (metastatic or locally advanced) disease. The current standard of care for unresectable or metastatic pancreatic cancer is palliative systemic chemotherapy with either gemcitabine (Gem) monotherapy, gemcitabine/nab-paclitaxel, or folinic acid/fluorouracil/irinotecan/oxaliplatin (FOLFIRINOX). For patients with borderline resectable or locally advanced disease, combination regimens have been used to potentially convert some borderline resectable and even some locally advanced tumors to resectability. In addition, the relatively hypovascular tumor microenvironment seen in most pancreatic adenocarcinomas makes targeted and comprehensive arterial delivery of chemotherapeutic agents challenging using conventional techniques.

Further, locally advanced pancreatic ductal adenocarcinoma (LA-PDAC) is associated with rapid progression, resistance to conventional therapies, deterioration in quality of life, significant morbidity, and a high mortality rate. PDAC tumors are characterized by dense desmoplastic stroma with a paucity of effector immune cells, rendering both drug delivery and stimulation of immune responses very challenging.

Therefore, there remains a need in the art for a more accurate, better-localized methods of delivering chemotherapy to treat solid tumors, such as pancreatic cancer that can address the limitations of current techniques.

### SUMMARY OF THE INVENTION

The present invention relates to methods of treating cancer and methods of delivering TLR agonists to solid tumors in the pancreas using a locoregional therapy through the vasculature.

In another aspect, the present invention relates to a method of treating pancreatic cancer comprising administering a TLR agonist through an intravascular device by pancreatic retrograde venous infusion (PRVI). According to another embodiment, the treatment of pancreatic cancer comprises administering a TLR agonist through an intravascular device by pancreatic arterial infusion (PAI).

In some embodiments, the TLR agonists are administered through pressure-enabled drug delivery (PEDD), which includes the administration of a therapeutic through a device, such as a catheter device, which generates, causes, and/or contributes to a net increase in fluid pressure within the vessel and/or target tissue or tumor.

In some embodiments, the TLR agonists are administered through a pressure-enabled device, such as one that increases vascular pressure.

In some embodiments, the TLR agonist is a Class C type CpG oligodeoxynucleotide (CpG-C ODN).

In some embodiments, the administration of a TLR agonist through an intravascular device to the pancreas results in an enhancement to the responsiveness to checkpoint inhibitor therapy in the pancreatic cancer.

In some embodiments, the TLR agonist is a TLR9 agonist.

These and other objects, features, and advantages of the exemplary embodiments of the present disclosure will become apparent upon reading the following detailed description of the exemplary embodiments of the present disclosure, when taken in conjunction with the appended paragraphs.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure.

FIG. 1 illustrates the structure of SD-101.

FIGS. 2A-2B compare tumor volume after systemic saline infusion, saline infusion via PRVI/PEDD, systemic SD-101 infusion, and SD-101 infusion via PRVI/PEDD in a murine model, and contains data and in chart form, respectively.

FIGS. 3A-3B compare tumor weight after systemic saline infusion, saline infusion via PRVI/PEDD, systemic SD-101 infusion, and SD-101 infusion via PRVI/PEDD in a murine model, and contains data and in chart form, respectively.

FIG. 4 illustrates normalized labeled SD-101 signal intensity in porcine pancreas infused via PRVI comparing local concentration of SD-101 relative to adjacent non-target tissues within the pancreas (all data).

FIG. 5 illustrates normalized labeled SD-101 signal intensity in porcine pancreas infused via PRVI comparing local concentration of SD-101 relative to adjacent non-target tissues within the pancreas (with outlier removed).

FIG. 6 illustrates treated tissue volume for a SEAL Device as compared to the end hole catheter in a porcine model (all data).

FIG. 7 illustrates treated signal intensity for the SEAL Device as compared to the end hole catheter in a porcine model (all data).

FIG. 8 illustrates treated tissue volume for a SEAL Device as compared to the end hole catheter in a porcine model (outlier data removed).

FIG. 9 illustrates treated signal intensity for the SEAL Device as compared to the end hole catheter in a porcine model (outlier data removed).

FIG. 10A-10B illustrate the distribution pattern of labeled SD-101 delivered by end hole catheter and SEAL device to porcine tissue, respectively.

FIG. 11 illustrates the overall design for a study of pancreatic retrograde venous infusions (PRVI) using a PEDD of a TLR9 agonist, SD-101, for response rates to CPI in patients with locally advanced PDAC.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION

The following description of embodiments provides non-limiting representative examples referencing numerals to particularly describe features and teachings of different aspects of the invention. The embodiments described should be recognized as capable of implementation separately, or in combination, with other embodiments from the description of the embodiments. A person of ordinary skill in the art reviewing the description of embodiments should be able to learn and understand the different described aspects of the invention. The description of embodiments should facilitate understanding of the invention to such an extent that other implementations, not specifically covered but within the knowledge of a person of skill in the art having read the description of embodiments, would be understood to be consistent with an application of the invention.

### Toll-Like Receptor Agonists

Toll-like receptors are pattern recognition receptors that can detect microbial pathogen-associated molecular patterns (PAMPs). TLR stimulation, such as TLR9 stimulation, can not only provide broad innate immune stimulation, but can also specifically address the dominant drivers of immunosuppression in the liver and the pancreas. TLR1-10 are expressed in humans and recognize a diverse variety of microbial PAMPs. In this regard, TLR9 can respond to unmethylated CpG-DNA, including microbial DNA. CpG refers to the motif of a cytosine and guanine dinucleotide linked by a phosphate backbone. TLR9 is constitutively expressed in B cells, plasmacytoid dendritic cells (pDCs), activated neutrophils, monocytes/macrophages, T cells, and MDSCs. TLR9 is also expressed in non-immune cells, including keratinocytes and gut, cervical, and respiratory epithelial cells. TLR9 can bind to its agonists in an intra-cellular compartment, within endosomes. Signaling may be carried out through MyD88/IkB/NfKB to induce pro-inflammatory cytokine gene expression. A parallel signaling pathway through IRF7 induces type 1 interferons (e.g., IFN-α, IFN-7, etc.) which stimulate adaptive immune responses. Further, TLR9 agonists can induce cytokine and IFN production and functional maturation of antigen presenting dendritic cells.

According to an embodiment, TLR9 stimulation can reduce and reprogram MDSCs. MDSCs are the key drivers of immunosuppression in the liver. MDSCs also drive expansion of other suppressor cell types such as Tregs, tumor-associated macrophages (TAMs), and cancer-associated fibroblasts (CAFs). MDSCs may shut down immune cells and immunotherapeutics. Further, high MDSC levels generally predict poor outcomes in cancer patients. In this regard, eliminating MDSCs is thought to improve the ability of the host's immune system to attack the cancer as well as the ability of the immunotherapy to induce deep responses. In an embodiment, TLR9s may convert MDSCs into immunostimulatory M1 macrophages, convert immature dendritic cells to mature dendritic cells, and expand effector T cells creating a responsive tumor microenvironment that may promote anti-tumor activity.

According to an embodiment, synthetic CpG-oligonucleotides (CPG-ONs) mimicking the immunostimulatory nature of microbial CpG-DNA can be developed for therapeutic use. According to an embodiment, the oligonucleotide is an oligodeoxynucleotide (ODN). There are a number of different CpG-ODN class types, e.g., Class A, Class B, Class C, Class P, and Class S, which share certain structural and functional features. In this regard, Class A type CPG-ODNs (or CPG-A ODNs) are associated with pDC maturation with little effect on B cells as well as the highest degree of IFNa induction; Class B type CPG-ODNs (or CPG-B ODNs) strongly induce B-cell proliferation, activate pDC and monocyte maturation, NK cell activation, and inflammatory cytokine production; and Class C type CPG-ODNs (or CPG-C ODNs) can induce B-cell proliferation and IFN-α production. Further, according to an embodiment, CPG-C ODNs can be associated with the following attributes: (i) unmethylated dinucleotide CpG motifs, (ii) juxtaposed CpG motifs with flanking nucleotides (e.g., AACGTTCGAA), (iii) a complete phosphorothioate (PS) backbone that links the nucleotides (as opposed to the natural phosphodiester (PO) backbones found in bacterial DNA), and (iv) a self-complimentary, palindromic sequence (e.g., AACGTT). In this regard, CPG-C ODNs may bind themselves due to their palindromic nature, thereby producing double-stranded duplex or hairpin structures.

Further, according to an embodiment, the CPG-C ODNs can include one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In an embodiment, the CpG-C ODNs are 12 to 100 bases in length, preferably 12 to 50 bases in length, preferably 12 to 40 bases in length, or preferably 12-30 bases in length. In an embodiment, the CpG-C ODN is 30 bases in length. In an embodiment, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In an embodiment, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length.

In an embodiment, the at least one palindromic sequence is 8 to 97 bases in length, preferably 8 to 50 bases in length, or preferably 8 to 32 bases in length. In an embodiment, the at least one palindromic sequence is at least (lower limit) 8, 10, 12, 14, 16, 18, 20, 22, 24, 26, 28, or 30 bases in length. In an embodiment, the at least one palindromic sequence is at most (upper limit) 50, 48, 46, 44, 42, 40, 38, 36, 34, 32, 30, 28, 26, 24, 22, 20, 18, 16, 14, 12 or 10 bases in length.

In an embodiment, the CpG-C ODN can comprise the sequence of SEQ ID NO: 1.

According to an embodiment, the CpG-C ODN can comprise the SD-101. SD-101 is a 30-mer phosphorothioate oligodeoxynucleotide, having the following sequence:

(SEQ ID NO: 1)
5′-TCG AAC GTT CGA ACG TTC GAA CGT TCG AAT-3′

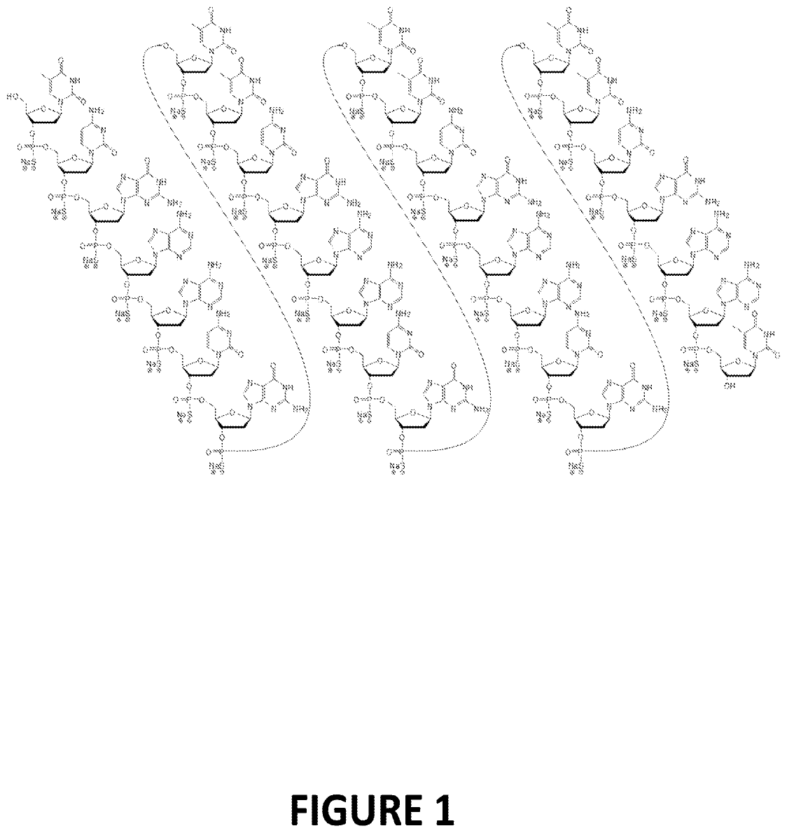

SD-101 drug substance is isolated as the sodium salt. The structure of SD-101 is illustrated in FIG. 1. The molecular formula of SD-101 free acid is C₂₉₃ H₃₆₉ N₁₁₂ O₁₄₉ P₂₉ S₂₉ and the molecular mass of the SD-101 free acid is 9.672 Daltons. The molecular formula of SD-101 sodium salt is C₂₉₃ H₃₄₀N₁₁₂ O₁₄₉ P₂₉ S₂₉ Na₂₉ and the molecular mass of the SD-101 sodium salt is 10,309 Daltons.

Further, according to an embodiment, the CPG-C ODN sequence can correspond to SEQ ID NO 172 as described in U.S. Pat. No. 9,422,564, which is incorporated by reference herein in its entirety.

In an embodiment, the CpG-C ODN can comprise a sequence that has at least 75% homology to any of the foregoing, such as SEQ ID NO:1.

According to another embodiment the CPG-C ODN sequence can correspond to any one of the other sequences described in U.S. Pat. No. 9,422,564. Further, the CPG-C ODN sequence can also correspond to any of the sequences described in U.S. Pat. No. 8,372,413, which is also incorporated by reference herein in its entirety.

According to an embodiment, any of the CPG-C ODNs discussed herein may be present in their pharmaceutically acceptable salt form. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In an embodiment, the CpG-C ODNs are in the ammonium, sodium, lithium, or potassium salt form. In one preferred embodiment, the CpG-C ODNs are in the sodium salt form. The CpG-C ODN may be provided in a pharmaceutical solution comprising a pharmaceutically acceptable excipient. Alternatively, the CpG-C ODN may be provided as a lyophilized solid, which is subsequently reconstituted in sterile water, saline or a pharmaceutically acceptable buffer before administration. Pharmaceutically acceptable excipients of the present disclosure include for instance, solvents, bulking agents, buffering agents, tonicity adjusting agents, and preservatives. In an embodiment, the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral and/or percutaneous administration.

In an embodiment, the pharmaceutical compositions comprise an aqueous vehicle as a solvent. Suitable vehicles include for instance sterile water, saline solution, phosphate buffered saline, and Ringer's solution. In an embodiment, the composition is isotonic.

The pharmaceutical compositions may comprise a bulking agent. Bulking agents are particularly useful when the pharmaceutical composition is to be lyophilized before administration. In an embodiment, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In an embodiment, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin, and mannitol.

The pharmaceutical compositions may comprise a preservative. Suitable preservatives include for instance antioxidants and antimicrobial agents. However, in an embodiment, the pharmaceutical composition is prepared under sterile conditions and is in a single use container, and thus does not necessitate inclusion of a preservative.

Table 1 describes the batch formula for SD-101 Drug Product 16 g/L:

TABLE 1

| | | | Amount | Clinical Lot | |
|---|---|---|---|---|---|
| | | Concen- | per | DVXA05 | |
| Ingredient | Grade | tration | batch | Batch Size 2.224 L | |
| SD-101 Drug | GMP | 1.6% | 16.00 g | 35.584 | g¹ |
| Substance* | | | | | |
| Sodium | USP/NF, | 1.02% | 1.02 g | 2.268 | g |
| phosphate, | EP | | | | |
| dibasic | | | | | |
| anhydrous | | | | | |
| Sodium | USP | 0.34% | 0.34 g | 0.747 | g |
| phosphate, | | | | | |
| monobasic | | | | | |
| anhydrous | | | | | |
| Sodium chloride | USP/NF, | 7.31% | 7.31 g | 16.257 | g |
| | EP | | | | |

| | | | | |
|---|---|---|---|---|
| Sterile Water for | USP/NF, | QS | QS | 2.224 L |
| Injection | EP | | | (kg) (QS) |

¹Quantity based upon measured content in solution (to exclude moisture present in lyophilized powder)
*SD-101 Drug Substance comprises the totality of all oligonucleotide content, including SD-101

In some embodiments, the unit dose strength may include from about 0.1 mg/mL to about 20 mg/mL. In one embodiment, the unit dose strength of SD-101 is 13.4 mg/mL.

CpG-C ODNs may contain modifications. Suitable modifications can include but are not limited to, modifications of the 3′OH or 5′OH group, modifications of the nucleotide base, modifications of the sugar component, and modifications of the phosphate group. Modified bases may be included in the palindromic sequence as long as the modified base(s) maintains the same specificity for its natural complement through Watson-Crick base pairing (e.g., the palindromic portion of the CpG-C ODN remains self-complementary).

CpG-C ODNs may be linear, may be circular or include circular portions and/or a hairpin loop. CpG-C ODNs may be single stranded or double stranded. CpG-C ODNs may be DNA, RNA or a DNA/RNA hybrid.

CpG-C ODNs may contain naturally-occurring or modified, non-naturally occurring bases, and may contain modified sugar, phosphate, and/or termini. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. In an embodiment, CpG-C ODNs have only phosphorothioate linkages, only phosphodiester linkages, or a combination of phosphodiester and phosphorothioate linkages.

Sugar modifications known in the field, such as 2′-alkoxy-RNA analogs, 2′-amino-RNA analogs, 2′-fluoro-DNA, and 2′-alkoxy- or amino-RNA/DNA chimeras and others described herein, may also be made and combined with any phosphate modification. Examples of base modifications include but are not limited to addition of an electron-withdrawing moiety to C-5 and/or C-6 of a cytosine of the CpG-C ODN (e.g., 5-bromocytosine, 5-chlorocytosine, 5-fluorocytosine, 5-iodocytosine) and C-5 and/or C-6 of a uracil of the CpG-C ODN (e.g., 5-bromouracil, 5-chlorouracil, 5-fluorouracil, 5-iodouracil). As noted above, use of a base modification in a palindromic sequence of a CpG-C ODN should not interfere with the self-complementarity of the bases involved for Watson-Crick base pairing. However, outside of a palindromic sequence, modified bases may be used without this restriction. For instance, 2′-O-methyl-uridine and 2′-O-methyl-cytidine may be used outside of the palindromic sequence, whereas, 5-bromo-2′-deoxycytidine may be used both inside and outside the palindromic sequence. Other modified nucleotides, which may be employed both inside and outside of the palindromic sequence include 7-deaza-8-aza-dG, 2-amino-dA, and 2-thio-dT.

Duplex (i.e., double stranded) and hairpin forms of most ODNs are often in dynamic equilibrium, with the hairpin form generally favored at low oligonucleotide concentration and higher temperatures. Covalent interstrand or intrastrand cross-links increase duplex or hairpin stability, respectively, towards thermal-, ionic-, pH-, and concentration-induced conformational changes. Chemical cross-links can be used to lock the polynucleotide into either the duplex or the hairpin form for physicochemical and biological characterization. Cross-linked ODNs that are conformationally homogeneous and are “locked” in their most active form (either duplex or hairpin form) could potentially be more active than their uncross-linked counterparts. Accordingly, some CpG-C ODNs of the present disclosure can contain covalent interstrand and/or intrastrand cross-links.

The techniques for making polynucleotides and modified polynucleotides are known in the art. Naturally occurring DNA or RNA, containing phosphodiester linkages, may be generally synthesized by sequentially coupling the appropriate nucleoside phosphoramidite to the 5′-hydroxy group of the growing ODN attached to a solid support at the 3′-end, followed by oxidation of the intermediate phosphite triester to a phosphate triester. Using this method, once the desired polynucleotide sequence has been synthesized, the polynucleotide is removed from the support, the phosphate triester groups are deprotected to phosphate diesters and the nucleoside bases are deprotected using aqueous ammonia or other bases.

The CpG-C ODN may contain phosphate-modified oligonucleotides, some of which are known to stabilize the ODN. Accordingly, some embodiments include stabilized CpG-C ODNs. The phosphorous derivative (or modified phosphate group), which can be attached to the sugar or sugar analog moiety in the ODN, can be a monophosphate, diphosphate, triphosphate, alkylphosphonate, phosphorothioate, phosphorodithioate, phosphoramidate or the like.

CpG-C ODNs can comprise one or more ribonucleotides (containing ribose as the only or principal sugar component), deoxyribonucleotides (containing deoxyribose as the principal sugar component), modified sugars or sugar analogs. Thus, in addition to ribose and deoxyribose, the sugar moiety can be pentose, deoxypentose, hexose, deoxyhexose, glucose, arabinose, xylose, lyxose, and a sugar analog cyclopentyl group. The sugar can be in pyranosyl or in a furanosyl form. In the CpG-C oligonucleotide, the sugar moiety is preferably the furanoside of ribose, deoxyribose, arabinose or 2′-0-alkylribose, and the sugar can be attached to the respective heterocyclic bases either in anomeric configuration. The preparation of these sugars or sugar analogs and the respective nucleosides wherein such sugars or analogs are attached to a heterocyclic base (nucleic acid base) per se is known, and therefore need not be described here. Sugar modifications may also be made and combined with any phosphate modification in the preparation of a CpG-C ODN.

The heterocyclic bases, or nucleic acid bases, which are incorporated in the CpG-C ODN can be the naturally-occurring principal purine and pyrimidine bases, (namely uracil, thymine, cytosine, adenine and guanine, as mentioned above), as well as naturally-occurring and synthetic modifications of said principal bases. Thus, a CpG-C ODN may include one or more of inosine, 2′-deoxyuridine, and 2-amino-2′-deoxyadenosine.

According to another embodiment, the CPG-ODN is one of a Class A type CPG-ODNs (CPGP-A ODNs), a Class B type CPG-ODNs (CPG-B ODNs), a Class P type CPG-ODNs (CPG-P ODN), and a Class S type CPG-ODNs (CPG-S ODN). In this regard, the CPG-A ODN can be CMP-001.

In another embodiment, the CPG-ODN can be tilsotolimod (IMO-2125).

### Checkpoint Inhibitors

According to an embodiment, the checkpoint inhibitor can include a Programmed Death 1 receptor (PD-1) antagonist. A PD-1 antagonist can be any chemical compound or biological molecule that blocks binding of Programmed Cell Death 1 Ligand 1 (PD-L1) expressed on a cancer cell to PD-1 expressed on an immune cell (T cell, B cell or NKT cell) and preferably also blocks binding of PD-L2 Programmed Cell Death 1 Ligand 2 (PD-L2) expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1.

According to an embodiment, the PD-1 antagonist can include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

According to an embodiment, the PD-1 antagonist can include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule.

According to an embodiment, the PD-1 antagonist can inhibit the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain.

According to an embodiment, the PD-1 antagonist can be one of nivolumab, pembrolizumab, and cemiplimab.

According to another embodiment, pembrolizumab is administered intravenously (IV) via a peripheral vein at a dose of 200 mg every three weeks (“Q3W”). In yet another embodiment, pembrolizumab is administered concomitantly, at the same time, at about the same time, or on the same day with SD-101. In another embodiment, pembrolizumab is administered one a weekly, every other week, every three weeks, every four weeks, or on a monthly basis following the administration of one or more cycles of SD-101. In another embodiment, pembrolizumab is administered for a period of up to six months.

According to another embodiment, nivolumab is administered intravenously (IV) via a peripheral vein at a dose of 240 mg every two weeks (“Q2W”). In yet another embodiment, nivolumab is administered concomitantly, at the same time, at about the same time, or on the same day with SD-101. In another embodiment, nivolumab is administered one a weekly, every other week, every three weeks, every four weeks, or on a monthly basis following the administration of one or more cycles of SD-101.

According to another embodiment, the checkpoint inhibitor can include a PD-L1 antagonist. In this regard, the PD-L1 antagonist can be one of atezolizumab, avelumab, and durvalumab.

According to another embodiment, the CPI can include a CTLA-4 antagonist. In this regard, the CTLA-4 antagonist can be ipilimumab. According to another embodiment, ipilimumab is administered intravenously (IV) via a peripheral vein at a dose of 3 mg/kg every three weeks. In yet another embodiment, ipilimumab is administered concomitantly, at the same time, at about the same time, or on the same day with SD-101. In another embodiment, nivolumab is administered one a weekly, every other week, every three weeks, every four weeks, or on a monthly basis following the administration of one or more cycles of SD-101.

### Devices to Achieve Locoregional Delivery

According to an embodiment, any of the above-described devices may comprise any device useful to achieve locoregional delivery to a tumor, including a catheter itself, or may comprise a catheter along with other components (e.g., filter valve, balloon, pressure sensor system, pump system, syringe, outer delivery catheter, etc.) that may be used in combination with the catheter. In certain embodiments, the catheter is a microcatheter.

In some embodiments, the device may have one or more attributes that include, but are not limited to, self-centering capability that can provide homogeneous distribution of therapy in downstream branching network of vessels; anti-reflux capability that can block or inhibit the retrograde flow of the TLR agonist (for example, with the use of a valve and filter, and/or balloon); a system to measure the pressure inside the vessel; and a means to modulate the pressure inside the vessel. In retrograde venous infusion, pressure in the vessel increases after device deployment which prevents retrograde flow. Infusion further increases vascular pressure in direct proportion to the rate of infusion. In arterial infusion, the deployment of the device reduces vascular pressure and flow. Infusion then increases vascular pressure in direct proportion to the rate of infusion. In some embodiments, the system is designed to continuously monitor real-time pressure throughout the procedure.

In some embodiments, the device that may be used to perform the methods of the present invention is a device as disclosed in U.S. Pat. Nos. 8,500,775, 8,696,698, 8,696,699, 9,539,081, 9,808,332, 9,770,319, 9,968,740, 10,813,739, 10,588,636, 11,090,460, U.S. Patent Publication No. 2018/0193591, U.S. Patent Publication No. 2018/0250469, U.S. Patent Publication No. 2019/0298983, U.S. Patent Publication No. 2020/0038586, and U.S. Patent Publication No. 2020-0383688, which are all incorporated by reference herein in their entireties.

In some embodiments, the device is a device as disclosed in U.S. Pat. No. 9,770,319. In certain embodiments, the device may be a device known as the Surefire Infusion System.

In some embodiments, the device supports the measurement of intravascular pressure during use. In some embodiments, the device is a device as disclosed in U.S. patent application Ser. No. 16/431,547. In certain embodiments, the device may be a device known as the TriSalus Infusion System (sometimes also known as the SEAL device). In certain embodiments, the device may be a device known as the TriNav® Infusion System. In certain embodiments, the device may be a device known as the SEAL Device. In certain embodiments, the catheter device may be described an anti-reflux microcatheter (TIS-21120-60) manufactured by TriSalus Life Sciences. In certain embodiment, the device may be a temporary occlusion device, such as the SEAL Device.

In some embodiments, the SEAL Device can be a dual catheter mechanically actuated infusion system equipped with a structure at the distal end of the device that acts to reversibly occlude blood flow in a retrograde venous infusion (RVI) procedure. According to an embodiment, the structure at the distal end of the device can be a braided filament construct with a fluid impermeable membrane provided over a proximal portion of the braided construct and a fluid permeable coating (or covering) over a distal portion of the braided construct. The device geometry may further allow for direct continuous pressure measurements of the vasculature distal to the device infusion lumen during therapeutic delivery. Device deployment and the infusion of therapeutic may modulate distal vascular pressure during RVI procedures.

In some embodiments, the TLR agonist may be administered through a device via PEDD. In some embodiments, the TLR agonist may be administered while monitoring the pressure in the vessel, which can be used to adjust and correct the positioning of the device at the infusion site and/or to adjust the rate of infusion. Pressure may be monitored by, for example, a pressure sensor system comprising one or more pressure sensors.

The rate of infusion may be adjusted to alter vascular pressure, which may promote the penetration of the TLR agonist into the target tissue or tumor. In some embodiments, the rate of infusion may be adjusted and/or controlled using a syringe pump as part of the delivery system. In some embodiments, the rate of infusion may be adjusted and/or controlled using a pump system. In some embodiments, the rate of infusion may be about 0.1 cc/min to about 40 cc/min, or about 0.1 cc/min to about 30 cc/min, or about 0.5 cc/min to about 25 cc/min, or about 0.5 cc/min to about 20 cc/min, or about 1 cc/min to about 15 cc/min, or about 1 cc/min to about 10 cc/min, or about 1 cc/min to about 8 cc/min, or about 1 cc/min to about 5 cc/min.

The present invention will be further illustrated and/or demonstrated in the following Examples, which is given for illustration/demonstration purposes only and is not intended to limit the invention in anyway.

### Methods Comprising Administration to the Pancreas

In an embodiment, the methods of the present invention include methods of treating pancreatic cancer, said method comprising administering a toll-like receptor agonist to a patient in need thereof, wherein the toll-like receptor agonist is administered through a device by PRVI to a solid tumor in the pancreas. PRVI refers to the infusion of a treatment to a solid tumor in the pancreas via a branch or branches of the pancreatic venous drainage system. According to an embodiment, the toll-like receptor agonists are introduced through the percutaneous transhepatic introduction of a device into the branch(es) of the pancreatic venous drainage system, such as a catheter and/or a device that facilitates pressure-enabled delivery. According to an embodiment, the toll-like receptor agonist is a TLR9 agonist and in some embodiments the TLR9 agonist is SD-101. In one embodiment, the patient is a human patient.

In an embodiment, delivery of the treatment by PRVI can be a more effective route of providing the TLR9 agonists to pancreatic tumors. In particular, in contrast to systemic intravenous and locoregional intra-arterial therapies, PRVI can be used to provide treatment to the tumor without relying on the arterial supply to the tumor, and, therefore may be a more effective means of delivering the TLR9 agonists and treating pancreatic cancer. For example, with PRVI, the TLR9 agonists can be delivered to the tumor via a sub-selective, catheter-directed approach utilizing the draining veins of the targeted pancreatic tumor. For example, the TLR9 agonist can be delivered to the tumor in a branch or branches of the pancreatic venous drainage system. In this regard, a digital subtraction angiography with computed tomography (CT) can be used to catheterize the veins draining the pancreatic tumor with a delivery device (e.g., catheter and/or a device that facilitates pressure-enabled delivery) in order to deliver the TLR9 agonists in a retrograde fashion.

In an embodiment, the methods of the present invention include methods of treating pancreatic cancer, said method comprising administering a toll-like receptor agonist to a patient in need thereof, wherein the toll-like receptor agonist is administered through a device by infusion through the pancreatic arterial system to a solid tumor in the pancreas. According to an embodiment, the toll-like receptor agonists are introduced through the percutaneous introduction of a device into the pancreatic arterial system, such as a catheter and/or a device that facilitates pressure-enabled delivery. For example, the pancreatic arterial system can be accessed by means of the splenic artery, the gastroduodenal artery, or the inferior pancreatic duodenal artery. In this regard, the head can be accessed through the gastroduodenal artery to the anterior and posterior pancreatic duodenal arteries, while the body and tail can be accessed from the splenic artery to the dorsal pancreatic artery, the great pancreatic artery, or the caudal pancreatic artery. From these vessels, smaller feeding vessels can be selected as required for the treatment of the target tissue. According to an embodiment, the toll-like receptor agonist is a TLR9 agonist and in some embodiments the TLR9 agonist is SD-101. In one embodiment, the patient is a human patient.

The pancreatic cancer can comprise a solid tumor in the pancreas, such as an exocrine tumor, such as a pancreatic adenocarcinoma, or endocrine tumor, such as neuroendocrine cancer. Examples include, but are not limited to, ductal adenocarcinoma (including pancreatic ductal adenocarcinoma and locally advanced pancreatic ductal adenocarcinoma) and acinar adenocarcinoma. In an embodiment, the tumor is unresectable or resection is not a reasonable undertaking due to the presence of advanced disease. Further, in an embodiment, the tumor is a metastatic pancreatic adenocarcinoma.

According to one embodiment, the methods of the present invention include a method for treating pancreatic adenocarcinoma, wherein the subject is eighteen years of age or older and exhibits histologically or cytologically confirmed evaluable or measurable locally advanced unresectable PDAC according to RECIST v1.1 criteria. In another embodiment, imaging confirmation centrally of unresectable disease as defined by NCCN occurs. In an additional embodiment, methods of the present invention may include administration to a subject who exhibits an Eastern Cooperative Oncology Group (“ECOG”) performance score (“PS”) of 0-1. In another embodiment, methods of the present invention may include administration to a subject exhibiting suitable venous anatomy on CT venogram as defined by absence of portal, splenic, or superior mesenteric vein complete occlusion.

According to another embodiment, the methods of the present invention include a method for treating pancreatic adenocarcinoma, wherein the subject has received standard of care chemoradiation therapy or a systemic chemotherapy regimen without a complete radiographic response. Examples of standard of care chemotherapy include gemcitabine+nab-paclitaxel, or FOLFIRINOX. In addition, radiation with or without concurrent chemotherapy is also acceptable as a standard of care regimen. In another embodiment, the subject has not received prior cytotoxic chemotherapy, targeted therapy, or external radiation therapy within 14 days prior to screening.

According to another embodiment, the methods of the present invention include a method for treating pancreatic adenocarcinoma wherein the subject has adequate hematologic and organ function. In another embodiment, the subject has no prior history of or other concurrent malignancy unless the malignancy is clinically insignificant, no ongoing treatment is required, and the subject is clinically stable. In another embodiment, the subject has measurable disease in the liver according to RECIST v.1.1 criteria.

According to another embodiment, methods of the present invention include a method for treating pancreatic adenocarcinoma wherein the subject has life expectancy of greater than 3 months as estimated by the investigator. According to yet another embodiment, the subject has a QTc interval of ≤480 msec.

In another embodiment, all associated clinically significant drug-related toxicity from previous cancer therapy is resolved prior to treatment. In this embodiment, resolution is to Grade ≤1 or the patient's pretreatment level. In an additional embodiment, the subject may have Grade 2 alopecia and endocrinopathies controlled on replacement therapy.

In another embodiment, methods of the present invention may include administration to a subject who has adequate organ function at screening. In an embodiment, a subject with adequate organ function may exhibit one or more of the following: (i) platelet count >100,000/μL, (2) hemoglobin ≥8.0 g/dL, (3) white blood cell count (WBC) >2,000/μL (4) Serum creatinine 2.0 mg/dL unless the measured creatinine clearance is >30 mL/min calculated by Cockcroft-Gault formula, (5) total and direct bilirubin ≤2.0× the upper limit of normal (ULN) and alkaline phosphatase ≤5×ULN, (6) for patients with documented Gilbert's disease, total bilirubin up to 3.0 mg/dL, (7) ALT and AST ≤5×ULN, and (8) amylase and lipase ≤3×ULN, and (8) prothrombin time/International Normalized Ratio (INR) or activated partial thromboplastin time (aPTT) test results at screening 1.5×ULN (this applies only to patients who do not receive therapeutic anticoagulation; patients receiving therapeutic anticoagulation should be on a stable dose for at least 4 weeks prior to the first dose of study intervention).

According to another embodiment, the tumor is unresectable.

According to another embodiment, the methods of the present invention can be administered with other cancer therapeutics such as immuno-modulators, tumor-killing agents, and/or other targeted therapeutics.

According to an embodiment, TLR9 therapy can enable cell therapy by modulation of the immune system.

In one embodiment, the above methods of administration to the pancreas results in the penetration of the toll-like receptor agonist throughout the solid tumor, through the entire tumor, or through substantially the entire tumor. In an embodiment, such methods enhance perfusion of the toll-like receptor agonist to a patient in need thereof, including by overcoming interstitial fluid pressure and solid stress. In an embodiment, such methods enable delivery of the toll-like receptor to areas of the tumor that are not accessible to systemic circulation. In another embodiment, such methods deliver higher concentrations of the toll-like receptor agonist into such a tumor with less toll-like receptor agonist delivered to non-target tissue compared to other therapies, such as conventional systemic delivery via a peripheral vein, or via direct intertumoral injection. In one embodiment, such methods result in the reduction in size, growth rate, or elimination of the solid tumor.

In some embodiments, doses of a TLR9 agonist, such as SD-101 may be about 0.01 mg, about 0.03 mg, about 0.05 mg, about 0.1 mg, about 0.3 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, and about 12 mg. In some embodiments, SD-101 is administered at doses of 16 mg and 20 mg. Administration of a milligram amount of SD-101 (e.g. about 2 mg) describes administering about 2 mg of the composition illustrated in FIG. 1. For example, such an amount of SD-101 (e.g. about a 2 mg amount) may also exist within a composition that contains material in addition to such amount of SD-101, such as other related and unrelated compounds. Equivalent molar amounts of other pharmaceutically acceptable salts are also contemplated.

In some embodiments, doses of a TLR9 agonist, such as SD-101 may be between about 0.01 mg and about 12 mg, between about 0.01 mg and 10 mg, between about 0.01 mg and about 8 mg, and between about 0.01 mg and 4 mg. In some embodiments, doses of a TLR9 agonist, such as SD-101 may be between about 2 mg and about 12 mg, 2 mg and about 10 mg, between about 2 mg and about 8 mg, and between about 2 mg and 4 mg. In some embodiments, doses of a TLR9 agonist, such as SD-101 may be less than about 12 mg, less than about 10 mg, less than about 8 mg, less than about 4 mg, or less than about 2 mg. Such doses may be administered daily, weekly, or every other week. In one embodiment, doses of SD-101 are incrementally increased, such as through administration of about 0.5 mg, followed by about 2 mg, followed by about 4 mg, followed by about 8 mg, and then followed by about 12 mg.

In some embodiments, the methods of the present invention may comprise administering a dosing regimen comprising cycles, in which one or more of the cycles comprise administering SD-101 via PRVI and PEDD. As used herein, a “cycle” is a repeat of a dosing sequence. In one embodiment, one cycle comprises one dose per cycle. In one embodiment, a cycle of treatment according to the present invention may comprise periods of SD-101 administration followed by “off” periods or rest periods. In another embodiment, in addition to a single dose per cycle, the cycle further comprises one week, two weeks, three weeks, four weeks, or twenty-eight days as a rest period following the weekly administration of SD-101. In another embodiment, the dosing regimen comprises at least one, at least two, or at least three cycles, or longer. In another embodiment, treatment comprises administration over two cycles, with one dose per cycle and each cycle being one month apart.

In some embodiments, the present invention relates to the use of a TLR9 agonist in the manufacture of a medicament for treating a solid tumor in the pancreas, such as locally-advanced pancreatic ductal adenocarcinoma, said method comprising administering the TLR9 agonist to a patient in need thereof, wherein the TLR9 agonist is administered through a device by PRVI to such solid tumor in the pancreas.

In some embodiments, SD-101 is administered for the treatment of locally advanced pancreatic ductal adenocarcinoma at a dose of 0.5 mg through PRVI, and in some embodiments, the SD-101 is further administered through a device that modulates pressure (i.e. PEDD). In some embodiments, SD-101 is administered at a dose of 0.5 mg through PRVI through a device that modulates vascular pressure in combination with a checkpoint inhibitor, wherein the checkpoint inhibitor is pembrolizumab. In some embodiments, SD-101 is administered at a dose of 0.5 mg through PRVI through a device that modulates vascular pressure in combination with a checkpoint inhibitor, wherein the checkpoint inhibitor is nivolumab. In some embodiments, SD-101 is administered at a dose of 0.5 mg through PRVI through a device that modulates vascular pressure in combination with a checkpoint inhibitor, wherein the checkpoint inhibitor is ipilimumab. In some embodiments, SD-101 is administered at a dose of 0.5 mg through PRVI and through a device that modulates pressure in combination with pembrolizumab, nivolumab, and ipilimumab.

In some embodiments, SD-101 is administered for the treatment of locally advanced pancreatic ductal adenocarcinoma at a dose of 2 mg through PRVI, and in some embodiments, the SD-101 is further administered through a device that modulates pressure (i.e. PEDD). In some embodiments, SD-101 is administered at a dose of 2 mg through PRVI through a device that modulates vascular pressure in combination with a checkpoint inhibitor, wherein the checkpoint inhibitor is pembrolizumab. In some embodiments, SD-101 is administered at a dose of 2 mg through PRVI through a device that modulates vascular pressure in combination with a checkpoint inhibitor, wherein the checkpoint inhibitor is nivolumab. In some embodiments, SD-101 is administered at a dose of 2 mg through PRVI through a device that modulates vascular pressure in combination with a checkpoint inhibitor, wherein the checkpoint inhibitor is ipilimumab. In some embodiments, SD-101 is administered at a dose of 2 mg through PRVI and through a device that modulates pressure in combination with pembrolizumab, nivolumab, and ipilimumab.

In some embodiments, SD-101 is administered for the treatment of locally advanced pancreatic ductal adenocarcinoma at a dose of 4 mg through PRVI, and in some embodiments, the SD-101 is further administered through a device that modulates pressure (i.e. PEDD). In some embodiments, SD-101 is administered at a dose of 4 mg through PRVI through a device that modulates vascular pressure in combination with a checkpoint inhibitor, wherein the checkpoint inhibitor is pembrolizumab. In some embodiments, SD-101 is administered at a dose of 4 mg through PRVI through a device that modulates vascular pressure in combination with a checkpoint inhibitor, wherein the checkpoint inhibitor is nivolumab. In some embodiments, SD-101 is administered at a dose of 4 mg through PRVI through a device that modulates vascular pressure in combination with a checkpoint inhibitor, wherein the checkpoint inhibitor is ipilimumab. In some embodiments, SD-101 is administered at a dose of 4 mg through PRVI and through a device that modulates pressure in combination with pembrolizumab, nivolumab, and ipilimumab.

In some embodiments, SD-101 is administered for the treatment of locally advanced pancreatic ductal adenocarcinoma at a dose of 8 mg through PRVI, and in some embodiments, the SD-101 is further administered through a device that modulates pressure (i.e. PEDD). In some embodiments, SD-101 is administered at a dose of 8 mg through PRVI through a device that modulates vascular pressure in combination with a checkpoint inhibitor, wherein the checkpoint inhibitor is pembrolizumab. In some embodiments, SD-101 is administered at a dose of 8 mg through PRVI through a device that modulates vascular pressure in combination with a checkpoint inhibitor, wherein the checkpoint inhibitor is nivolumab. In some embodiments, SD-101 is administered at a dose of 8 mg through PRVI through a device that modulates vascular pressure in combination with a checkpoint inhibitor, wherein the checkpoint inhibitor is ipilimumab. In some embodiments, SD-101 is administered at a dose of 8 mg through PRVI and through a device that modulates pressure in combination with pembrolizumab, nivolumab, and ipilimumab.

In some embodiments, SD-101 is administered for the treatment of locally advanced pancreatic ductal adenocarcinoma at a dose of 12 mg through PRVI, and in some embodiments, the SD-101 is further administered through a device that modulates pressure (i.e. PEDD). In some embodiments, SD-101 is administered at a dose of 12 mg through PRVI through a device that modulates vascular pressure in combination with a checkpoint inhibitor, wherein the checkpoint inhibitor is pembrolizumab. In some embodiments, SD-101 is administered at a dose of 12 mg through PRVI through a device that modulates vascular pressure in combination with a checkpoint inhibitor, wherein the checkpoint inhibitor is nivolumab. In some embodiments, SD-101 is administered at a dose of 12 mg through PRVI through a device that modulates vascular pressure in combination with a checkpoint inhibitor, wherein the checkpoint inhibitor is ipilimumab. In some embodiments, SD-101 is administered at a dose of 12 mg through PRVI and through a device that modulates pressure in combination with pembrolizumab, nivolumab, and ipilimumab.

In some embodiments, the methods of the present invention comprise a step that allows the infusion to dwell in the affected tissue, such as the pancreas, for varying amounts of time. For example, methods of the present invention include dwell times of between about zero to about twenty minutes. In another embodiment, the methods of the present invention comprise a dwell time of about five to about ten minutes.

In some embodiments, the methods of the present invention result in the treatment of target lesions. In this embodiment, the methods of the present invention may result in a complete response, comprising the disappearance of all target lesions. In some embodiments, the methods of the present invention may result in a partial response, comprising at least a 30% decrease in the sum of the longest diameter of target lesions, taking as reference the baseline sum longest diameter. In some embodiments, the methods of the present invention may result in stable disease of target lesions, comprising neither sufficient shrinkage to qualify for partial response nor sufficient increase to qualify for progressive disease, taking as reference the smallest sum longest diameter since the treatment started. In such an embodiment, progressive disease is characterized by at least a 20% increase in the sum of the longest diameter of target lesions, taking as reference the smallest sum longest diameter recorded since the treatment started or the appearance of 1 or more new lesions. The sum must demonstrate an absolute increase on 5 mm.

In another embodiment, the methods of the present invention result in the treatment of non-target lesions. In this embodiment, the methods of the present invention may result in a complete response, comprising the disappearance of all nontarget lesions. In some embodiments, the methods of the present invention result in persistence of one or more nontarget lesion(s). In such an embodiment, progressive disease is characterized by unequivocal progression of existing nontarget lesions, and/or the appearance of one or more new lesions.

In some embodiments, the methods of the present invention result in a beneficial overall response rate, such as an overall response rate according to RECIST v.1.1. In those embodiments, the methods of the present invention result in an overall response that is a complete response wherein the subject exhibits a complete response of target lesions, a complete response of nontarget lesions, and no new lesions. In other embodiments, the methods of the present invention result in an overall response that is a partial response, wherein the subject exhibits a complete response for target lesions, non-complete response and non-progressive disease for non-target lesions, and no new lesions. In other embodiments, the methods of the present invention result in an overall response that is a partial response, wherein the subject exhibits a partial response for target lesions, non-progressive disease for non-target lesions, and no new lesions. In another embodiment, the methods of the present invention result in an overall response that is stable disease wherein the subject exhibits stable disease of target lesions, non-progressive disease for non-target lesions, and no new lesions.

In some embodiments, the methods of the present invention result in an increased duration of overall response. In some embodiments, the duration of overall response is measured from the time measurement criteria are met for complete response or partial response (whichever is first recorded) until the first date that recurrent or progressive disease is objectively documented (taking as reference for progressive disease the smallest measurements recorded since the treatment started). The duration of overall complete response may be measured from the time measurement criteria are first met for complete response until the first date that progressive disease is objectively documented. In some embodiments, the duration of stable disease is measured from the start of the treatment until the criteria for progression are met, taking as reference the smallest measurements recorded since the treatment started, including the baseline measurements.

In yet other embodiments, the methods of the present invention result in improved overall survival rates. For example, overall survival may be calculated from the date of enrollment to the time of death. Patients who are still alive prior to the data cutoff for final efficacy analysis, or who dropout prior to study end, will be censored at the day they were last known to be alive.

In other embodiments, the methods of the present invention result in progression-free survival. For instance, progression-free survival may be calculated from the date of documenting relapse (or other unambiguous indicator of disease development), or date of death, whichever occurs first. Patients who have no documented relapse and are still alive prior to the data cutoff for final efficacy analysis, or who drop out prior to study end, will be censored at the date of the last radiological evidence documenting absence of relapse.

In some embodiments, the methods of the present invention result in a beneficial overall response rate, such as an overall response rate according to iRECIST. In another embodiment, the methods result in clinical benefit (e.g. complete response+partial response+stable disease). In another embodiment, the methods of the present invention result in improvements in the Eastern Cooperative Oncology Group Performance Status (ECOG PS) compared to baseline over time. In yet another embodiment, the methods of the present invention result in improvements in quality of life using the European Organization for the Research and Treatment of Cancer Quality of Life Questionnaire for Cancer (EORTC-QLQ-C30) instrument.

According to another embodiment, the methods of the present invention include a method for treating as locally-advanced pancreatic ductal adenocarcinoma, wherein the administration of SD-101 results in a reduction of tumor burden. In some embodiments, the tumor burden is reduced by about 10%, by about 20%, by about 30%, by about 40%, by about 50%, by about 60%, by about 70%, by about 80%, by about 90%, or by about 100%.

According to another embodiment, the methods of the present invention include a method for treating locally-advanced pancreatic ductal adenocarcinoma, wherein the administration of SD-101 results in a reduction of tumor progression. In some embodiments, tumor progression is reduced by about 10%, by about 20%, by about 30%, by about 40%, by about 50%, by about 60%, by about 70%, by about 80%, by about 90%, or by about 100%.

According to another embodiment, the methods of the present invention include a method for treating locally-advanced pancreatic ductal adenocarcinoma, wherein the administration of SD-101 reprograms the liver MDSC compartment to enable immune control of liver metastases and/or improves responsiveness to systemic anti-PD-1 therapy through elimination of MDSC. In some embodiments, the methods of the present invention are superior in controlling MDSC. In some embodiments, the methods of the present invention include a method for locally-advanced pancreatic ductal adenocarcinoma, wherein the administration of SD-101 reduces the frequency of MDSC cells (CD11b+Gr1+), monocytic MDSC (M-MDSC; CD11b+Ly6C+) cells, or granulocytic MDSC (G-MDSC; CD11b+LY6G+) cells. According to another embodiment, the methods of the present invention enhance M1 macrophages. According to yet another embodiment, the methods of the present invention decrease M2 macrophages.

In another embodiment, the methods of the present invention increase NFκB phosphorylation. In yet an additional embodiment, the methods of present invention increase IL-6. In another embodiment, the methods of the present invention increase IL10. In yet an additional embodiment, the methods of present invention increase IL-29. In another embodiment, the methods of the present invention increase IFNα. As a further embodiment, the methods of the present invention decrease STAT3 phosphorylation.

### Example 1

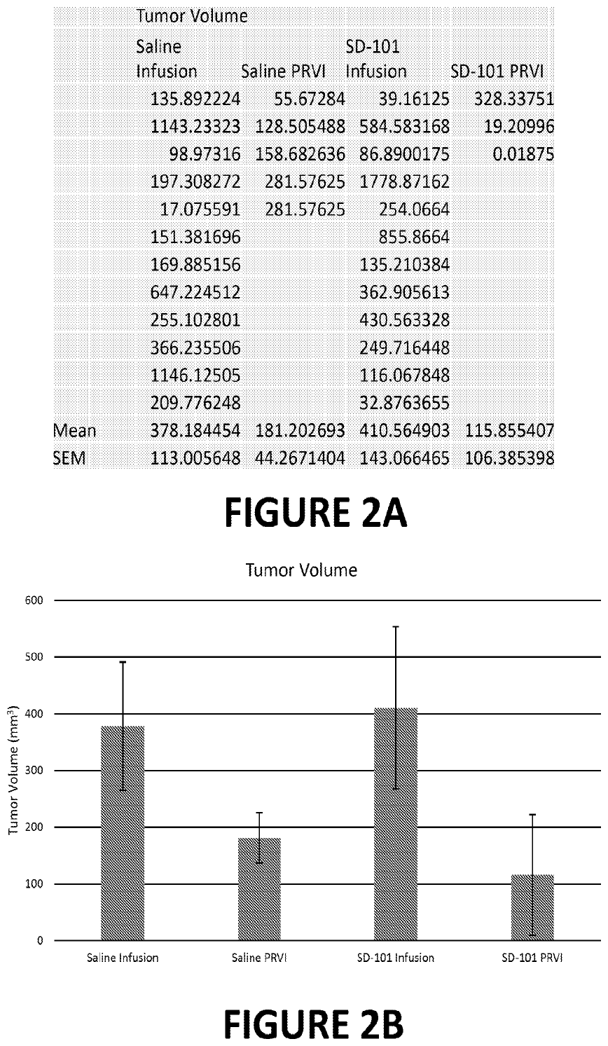

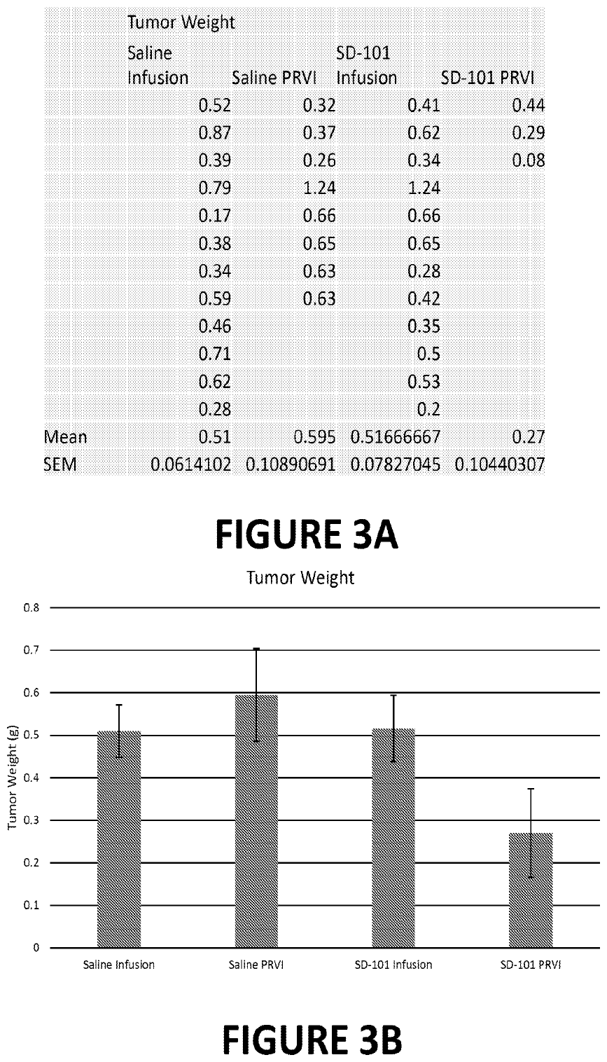

In this example, tumor volume and tumor weight were compared for systemic saline infusion, saline infusion via PRVI/PEDD, systemic SD-101 infusion, and SD-101 infusion via PRVI/PEDD in a murine model. FIG. 2A depicts the data for the tumor volume, while FIG. 2B depicts a chart illustrating the mean and the standard error of the mean (SEM) for the tumor volume. FIG. 3A depicts the data for the tumor weight, while FIG. 3B depicts a chart illustrating the mean and the SEM for the tumor weight.

As can be seen in the data and the corresponding figures, there is a trend toward improvement.

### Example 2

In the present example, a SD-101 sequence oligonucleotide was synthesized and conjugated to the IRDye800CW (ex. 767 nm, em. 791 nm) fluorophore.

/5IRD800CW/T*C*G*A*A*C*G*T*T*C*G*A*A*C*G*T*T*C*G*
A*A*C*G*T*T*C*G*A*A*T

The labeled SD-101 was then dissolved in saline solution and administered through the PEDD device (i.e., SEAL Device) in a porcine model at a rate of 2 ml/min. Blood was allowed to circulate for 60 min prior to euthanizing the animal and collection of the pancreatic tissue. NearIR imaging was employed to quantify signal intensity (a measure of labeled SD-101 concentration in the tissue) and treated tissue distribution. Untreated porcine pancreas was used as a reference to normalize signal intensity.

Three different experimental arms were conducted:
0 min dwell, 10 cc injection @ 2 cc/min with aspiration
20 min dwell, 10 cc injection @ 2 cc/min with aspiration
0 min dwell, 20 cc injection @ 2 cc/min without aspiration

In this analysis, any pig which showed a clear perforation of the vein and/or extravasation of fluid on fluoroscopy was excluded. There were five such occurrences noted in the data sheets. Each specimen is identified in Table 2 (Porcine Specimens in PRVI Study) including the treatment group, the date of the procedure, and the exclusion status.

TABLE 2

| | Specimen | | Dwell | Volume | |
|---|---|---|---|---|---|
| Date | # | Treatment | (min) | (cc) | Excluded? |
| Feb. 24, 2021 | P21040 | 1 | 0 | 10 | |
| | P21041 | 2 | 20 | 10 | |
| Mar. 10, 2021 | P21045 | 1 | 0 | 10 | |
| | P21046 | 2 | 20 | 10 | |
| Mar. 31, 2021 | P21061 | 1 | 0 | 10 | Y - small perforation at vein origin |
| | P21062 | 2 | 20 | 10 | |
| Apr. 6, 2021 | P21078 | 1 | 0 | 10 | Y - perforation |
| | P21079 | 2 | 20 | 10 | Y - perforation with contrast extravasation |
| Apr. 23, 2021 | P21088 | 1 | 0 | 10 | |
| Apr. 30, 2021 | P21089 | 1 | 0 | 10 | |
| | P21090 | 2 | 20 | 10 | |
| May 11, 2021 | P21092 | 1 | 0 | 10 | |
| | P21093 | 1 | 0 | 10 | |
| Jun. 30, 2021 | P21116 | 3 | 0 | 20 | Y - perforation |
| | P21117 | 3 | 0 | 20 | Y - placed two coils |
| Jul. 9, 2021 | P21112 | 3 | 0 | 20 | |
| Jul. 15, 2021 | P21126 | 3 | 0 | 20 | |
| | P21127 | 3 | 0 | 20 | |
| Jul. 29, 2021 | P21133 | 3 | 0 | 20 | |
| | P21134 | 3 | 0 | 20 | |

There were two exclusions from treatment 1, one from treatment 2, and two from treatment 3. As a result, 6 samples of treatment 1, 4 samples of treatment 2, and 5 samples of treatment 3 were included in this analysis.

The data analysis was completed on the Near TR images from these studies using MATLAB and the One-Channel-analysis V2 toolpak. Volume was calculated from the number of pixels. Signal was adjusted to the original dose measured from the syringe.

A summary of the data output is shown in Table 3 (Data Output from Analysis). It includes pressure measurements obtained from the Edwards Lifesciences pressure sensor and the Quantien pressure monitor.

TABLE 3

| | Specimen | | Volume | | Pressure | Pressure | Pressure |
|---|---|---|---|---|---|---|---|
| Date | # | Treatment | (cc) | Signal | Collapsed | Deployed | Peak |
| Feb. 24, 2021 | P21040 | 1 | 11.94 | 5,554 | 29 | 44 | 44 |
| Mar. 10, 2021 | P21045 | 1 | 2.53 | 459 | 15 | 33 | 32 |
| Apr. 23, 2021 | P21088 | 1 | 2.67 | 520 | 15 | 27 | 25 |
| Apr. 30, 2021 | P21089 | 1 | 5.71 | 2,276 | 15 | 24 | 32 |
| May 11, 2021 | P21092 | 1 | 0.50 | 95 | 25 | 33 | 40 |
| May 11, 2021 | P21093 | 1 | 11.43 | 9,279 | 25 | 35 | 35 |
| Feb. 24, 2021 | P21041 | 2 | 2.71 | 489 | 19 | 28 | 38 |
| Mar. 10, 2021 | P21046 | 2 | 4.56 | 960 | 12 | 31 | 41 |
| Mar. 31, 2021 | P21062 | 2 | 5.82 | 1,964 | 28 | 34 | 34 |
| Apr. 30, 2021 | P21090 | 2 | 5.94 | 1,240 | 19 | 35 | 45 |
| Jul. 9, 2021 | P21112 | 3 | 8.26 | 2,619 | 19 | 34 | 36 |
| Jul. 15, 2021 | P21126 | 3 | 4.85 | 765 | 30 | 31 | 34 |
| Jul. 15, 2021 | P21127 | 3 | 1.25 | 95 | 22 | 26 | 30 |
| Jul. 29, 2021 | P21133 | 3 | 5.54 | 2,758 | 22 | 35 | 45 |
| Jul. 29, 2021 | P21134 | 3 | 9.21 | 3,672 | 32 | 28 | 30 |

All statistics were performed using Minitab.

### Analysis of Variance

TABLE 4

| Source | DF | Adj SS | Adj MS | F-Value | P-Value |
|---|---|---|---|---|---|
| Treatment | 2 | 3.240 | 1.620 | 0.12 | 0.889 |
| Error | 12 | 164.093 | 13.674 | | |
| Total | 14 | 167.333 | | | |

No significant difference was found with p = .889.

A one-way ANOVA was performed on Signal comparing the three treatment groups with post-hoc Tukey pairwise comparisons.

### Analysis of Variance

TABLE 5

| Source | DF | Adj SS | Adj MS | F-Value | P-Value |
|---|---|---|---|---|---|
| Treatment | 2 | 9790602 | 4895301 | 0.69 | 0.521 |
| Error | 12 | 85331223 | 7110935 | | |
| Total | 14 | 95121824 | | | |

No significant difference was found with p = .521.

A linear regression was performed on both volume and signal using the treatment groups as the fixed variable and pressures as the continuous variables (collapsed, deployed, and/or peak) to see if pressure had an effect on either volume or signal.

### Analysis of Variance—Volume

TABLE 6

| Source | DF | Adj SS | Adj MS | F-Value | P-Value |
|---|---|---|---|---|---|
| Regression | 5 | 66.257 | 13.2514 | 1.18 | 0.390 |
| collapsed_pressure | 1 | 0.347 | 0.3473 | 0.03 | 0.864 |
| Deployed_pressure | 1 | 0.370 | 0.3705 | 0.03 | 0.860 |
| Peak_pressure | 1 | 25.537 | 25.5365 | 2.27 | 0.166 |
| Treatment | 2 | 2.192 | 1.0962 | 0.10 | 0.908 |
| Error | 9 | 101.076 | 11.2306 | | |
| Total | 14 | 167.333 | | | |

### Analysis of Variance—Signal

TABLE 7

| Source | DF | Adj SS | Adj MS | F-Value | P-Value |
|---|---|---|---|---|---|
| Regression | 5 | 44965015 | 8993003 | 1.61 | 0.251 |
| collapsed_pressure | 1 | 102665 | 102665 | 0.02 | 0.895 |
| Deployed_pressure | 1 | 520184 | 520184 | 0.09 | 0.767 |
| Peak_pressure | 1 | 21744188 | 21744188 | 3.90 | 0.080 |
| Treatment | 2 | 7122449 | 3561224 | 0.64 | 0.550 |
| Error | 9 | 50156810 | 5572979 | | |
| Total | 14 | 95121824 | | | |

No variables were found to be significant.

Table 8 summarizes some of the descriptive statistics.

TABLE 8

| | 0 min dwell, | 20 min dwell, | 0 min dwell, | |
|---|---|---|---|---|
| | 10 cc injection | 10 cc injection | 20 cc injection | Overall |
| Volume (cc) | 5.79 ± 4.86 | 4.75 ± 1.50 | 5.82 ± 3.14 | 5.53 ± 3.46 |
| Signal | 3167 ± 3841 | 1216 ± 645 | 1982 ± 1492 | 2239 ± 2607 |
| (no units) | | | | |
| Collapsed | 23 ± 6 | 17 ± 5 | 25 ± 6 | 22 ± 6 |
| Pressure | | | | |
| (mm Hg) | | | | |
| Deployed | 34 ± 6 | 30 ± 4 | 31 ± 4 | 32 ± 5 |
| Pressure | | | | |
| (mm Hg) | | | | |
| Peak Pressure | 37 ± 7 | 36 ± 5 | 35 ± 6 10 | 36 ± 6 |
| (mm Hg) | | | | |

A typical human pancreas is usually about 75 cc. One would expect a 50 kg pig to be similar or perhaps a little smaller. If one uses the MATLAB analysis and sums the total target area and total non-target area and calculate volume, this should equal the total organ volume. When this was done, the average across all pigs in this study is 73.62 cc. Therefore, this method appears to be an accurate way of determining the total pancreas volume. Table 9 summarizes the calculated organ volume.

TABLE 9

| Specimen | Organ Volume |
|---|---|
| P21040 | 91.68 |
| P21041 | 161.72 |
| P21045 | 63.95 |
| P21046 | 52.04 |
| P21062 | 65.35 |
| P21088 | 77.89 |
| P21089 | 73.86 |
| P21090 | 72.76 |
| P21092 | 32.64 |
| P21093 | 94.29 |
| P21112 | 85.15 |
| P21126 | 46.93 |
| P21127 | 46.03 |
| P21133 | 64.38 |
| P21134 | 75.63 |

The dose delivered was also quantified by the Near IR camera prior to delivery. This was done by taking images of the syringe after the dose is mixed with the saline and quantifying the signal. Table 10 summarizes the total dose delivered.

TABLE 10

| Specimen | Dose |
|---|---|
| P21040 | 372,333 |
| P21041 | 372,000 |
| P21045 | 343,000 |
| P21046 | 339,000 |
| P21062 | 375,000 |
| P21088 | 361,333 |
| P21089 | 365,667 |
| P21090 | 363,000 |
| P21092 | 378,333 |
| P21093 | 371,667 |
| P21112 | 442,667 |
| P21126 | 444,000 |
| P21127 | 440,667 |
| P21133 | 399,000 |
| P21134 | 341,000 |

The percentage of tissue coverage and dose delivered are as follows in Table 11.

TABLE 11

| | 0 min dwell, | 20 min dwell, | 0 min dwell, | |
|---|---|---|---|---|
| | 10 cc injection | 10 cc injection | 20 cc injection | Overall |
| % of Tissue | 7.0 ± 4.8% | 6.9 ± 3.5% | 8.7 ± 3.6% | 7.5 ± 3.9% |
| Covered | | | | |
| % of Dose | 0.86 ± 1.03% | 0.33 ± 0.17% | 0.51 ± 0.47% | 0.60 ± 0.71% |
| Delivered | | | | |

These two calculations indicated that an average of 0.6% of the total labeled SD-101 dose was absorbed by tissue comprising 7.5% of the pancreas volume. While this represents a small portion of the total dose, the local concentration of SD-101 in the target tissues was enriched relative to what the tissue would normally receive for systemic therapeutic delivery. As such, using a PRVI, a lower dosage of SD-101 can be used to achieve a similar uptake/response that can be done with a systemic infusion.

Further analysis was conducted without any procedural exclusions from the data set with the exception of P21116 and P21117 which did not receive PRVI infusion due to the inability to position the device after perforation. Data from all cohorts was pooled for analysis (n=18).

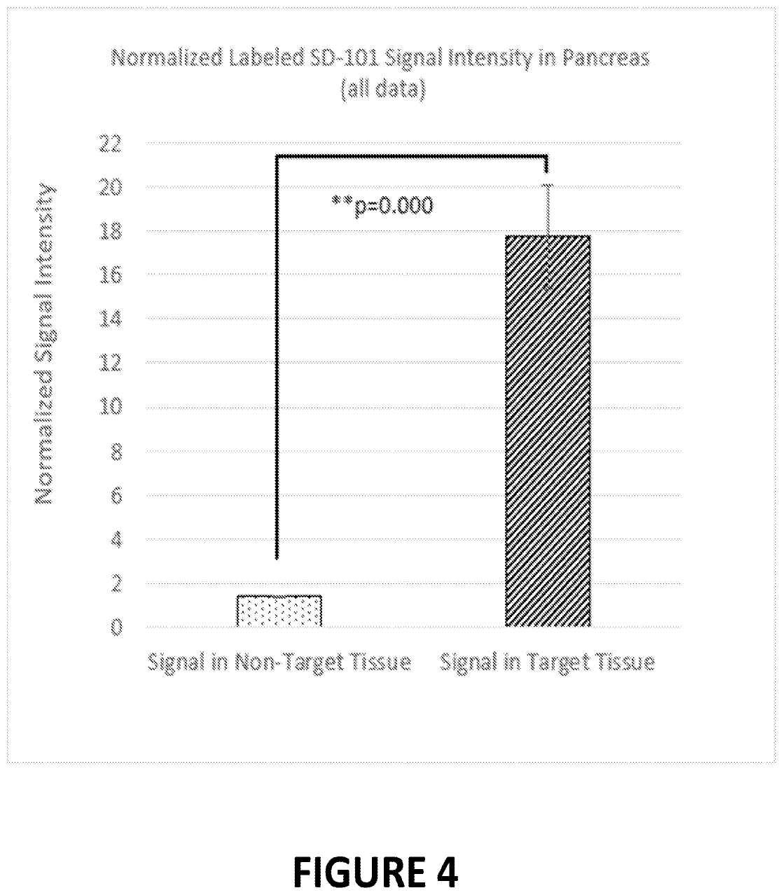

It was determined that PRVI raised local vascular pressure by 14±2 mmHg during infusion and increased local concentration of SD-101 by 12.6-fold relative to adjacent non-target tissues within the pancreas (17.12±2.39 target tissue vs 1.40±0.05 non-target tissue, p=0.000) (FIG. 4, Table 12). Tissue targeting by PRVI was found to be highly selective, with an average of 7.66% of the tissue volume exposed to labeled SD-101.

TABLE 12
Descriptive Statistics for Labeled SD-101 PRVI (with outlier)

| | |
|---|---|
| Organ volume (cc) | 76.73 ± 7.11 |
| Treated Volume (cc) | 5.87 ± 0.89 |
| Signal (raw intensity | 2264 ± 567 |
| measurement) (lu) | |
| Collapsed Pressure | 22 ± 1 |
| (mmHg) | |
| Deployed Pressure | 32 ± 1 |
| (mmHg) | |
| Peak Pressure | 36 ± 2 |
| (mmHg) | |

An outlier test was perform using MiniTab software. It was determined that the signal data from P21093 (9471 lu) was an outlier from the data set. The analysis was reanalyzed excluding data from P21093.

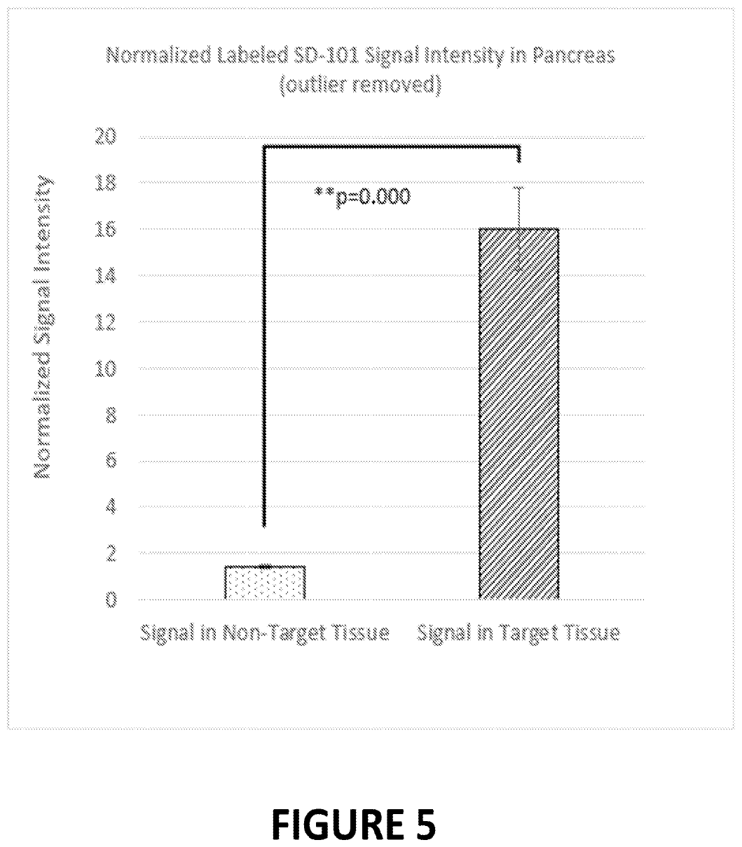

After removal of the outlier, it was determined that PRVI raised local vascular pressure by 13±2 mmHg during infusion and increased local concentration of SD-101 by 11.3-fold relative to adjacent non-target tissues within the pancreas (16.01±1.78 target tissue vs 1.41±0.06 non-target tissue, p=0.000) (FIG. 5, Table 13). Tissue targeting by PRVI was found to be highly selective, with an average of 7.33% of the tissue volume exposed to labeled SD-101.

TABLE 13
Descriptive Statistics for Labeled
SD-101 PRVI (with outlier removed)

| | |
|---|---|
| Organ volume (cc) | 75.70 ± 7.46 |
| Treated Volume (cc) | 5.55 ± 0.88 |
| Signal (raw intensity | 1840 ± 400 |
| measurement) (lu) | |
| Collapsed Pressure | 22 ± 1 |
| (mmHg) | |
| Deployed Pressure | 32 ± 1 |
| (mmHg) | |
| Peak Pressure | 36 ± 2 |
| (mmHg) | |

### Example 3

In this example, signal intensity (therapeutic absorption) and treated volume were compared for a PEDD device and an end hole catheter in a porcine model using IDR800CW labeled SD-101.

In this regard, there were two different experimental arms included in the following summary: (i) SEAL Device: 0 min dwell, 10 cc injection @ 2 cc/min with aspiration and (ii) an End hole catheter: 0 min dwell, 10 cc injection @ 2 cc/min without aspiration.

Data generated from the SEAL Device (0 min dwell, 10 cc injection @ 2 cc/min with aspiration) is depicted in Table 14.

TABLE 14
PRVI infusion of SD-101 with Seal Device

| | | | Pressure | Pressure | Pressure |
|---|---|---|---|---|---|
| | Volume | Signal | Collapsed | Deployed | Infusion |
| Animal ID | (cm³) | (lu) | (mmHg) | (mmHg) | (mmHg) |
| P21061 | 11.94 | 4148 | 27 | 41 | 59 |
| P21078 | 2.53 | 1294 | 21 | 24 | 24 |
| P21040 | 14.13 | 5554 | 29 | 44 | 44 |
| P21045 | 3.68 | 459 | 15 | 27 | 25 |
| P21088 | 2.67 | 519 | 25 | 35 | 35 |
| P21089 | 5.71 | 2276 | 19 | 28 | 38 |
| P21092 | 0.50 | 95 | 28 | 34 | 34 |
| P21093 | 11.43 | 9279 | 19 | 35 | 45 |
| Average | 6.57 | 2953 | 23 | 34 | 38 |
| Standard | 5.17 | 3201 | 5 | 7 | 11 |
| Deviation | | | | | |
| Standard Error | 1.83 | 1132 | 2 | 2 | 4 |

Data generated from the end hole Catheter (0 min dwell, 10 cc injection @2 cc/min without aspiration) is depicted in Table 15.

TABLE 15
PRVI infusion of SD-101 with end hole catheter

| | Volume | Signal | Pressure Proximal | Pressure Distal |
|---|---|---|---|---|
| Animal ID | (cm³) | (lu) | (mmHg) | (mmHg) |
| P21148 | 2.70 | 1123 | 16 | 16 |
| P21154 | 0.75 | 66 | 14 | 14 |
| P21147 | 1.60 | 163 | 14 | 14 |
| P21146 | 0.17 | 11 | 15 | 17 |
| P21159 | 0.13 | 23 | 11 | 11 |
| P21160 | 0.45 | 58 | 13 | 16 |
| Average | 0.97 | 241 | 14 | 15 |
| Standard | 1.01 | 435 | 2 | 2 |
| Deviation | | | | |
| Standard Error | 0.41 | 178 | 1 | 1 |

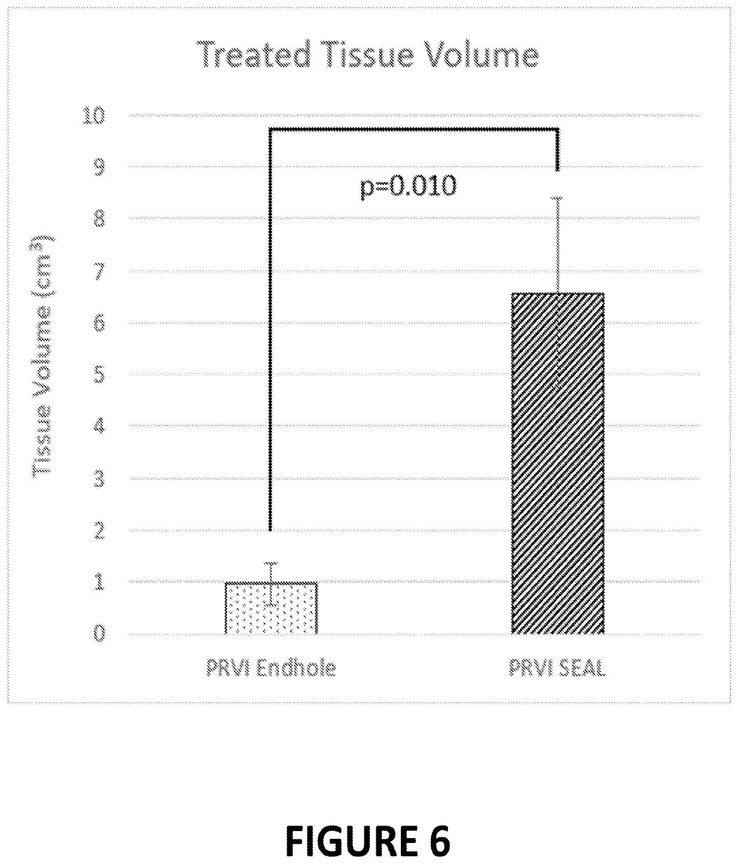

FIG. 6 depicts the treated volume for the SEAL Device as compared to the end hole catheter. In this regard, using the SEAL Device resulted in a 6.8 fold increase in treated tissue volume.

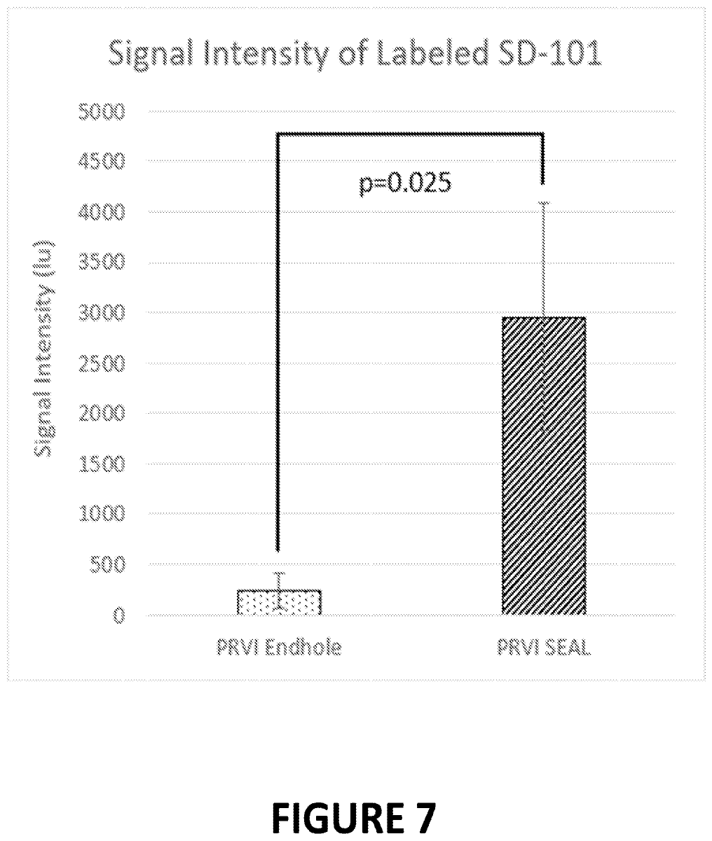

FIG. 7 depicts the treated signal intensity for the SEAL Device as compared to the end hole catheter. In this regard, using the SEAL Device resulted in a 12 fold increase in labeled SD-101 delivered to the tissue as measured by signal intensity.

As similar test was performed with the outlier removed. In this regard, Minitab software was used to determine if outliers were present in both the SEAL Device and end hole infusion data sets. This analysis determined that there was an outlier in the end hole Signal data set (P21148, 1122.88 lu signal intensity). The data was re-analyzed excluding data from P21148.

Table 16 depicts the end hole catheter data with the outlier removed.

TABLE 16
PRVI infusion of SD-101 with end hole Catheter

| | | | Pressure | Pressure |
|---|---|---|---|---|
| | Volume | Signal | Proximal | Distal |
| Animal ID | (cm³) | (lu) | (mmHg) | (mmHg) |
| P21154 | 0.75 | 66 | 14 | 14 |
| P21147 | 1.60 | 163 | 14 | 14 |
| P21146 | 0.17 | 11 | 15 | 17 |
| P21159 | 0.13 | 23 | 11 | 11 |
| P21160 | 0.45 | 58 | 13 | 16 |
| Average | 0.62 | 64 | 13.4 | 14.4 |
| Standard Deviation | 0.60 | 60 | 2 | 2 |
| Standard Error | 0.27 | 27 | 1 | 1 |

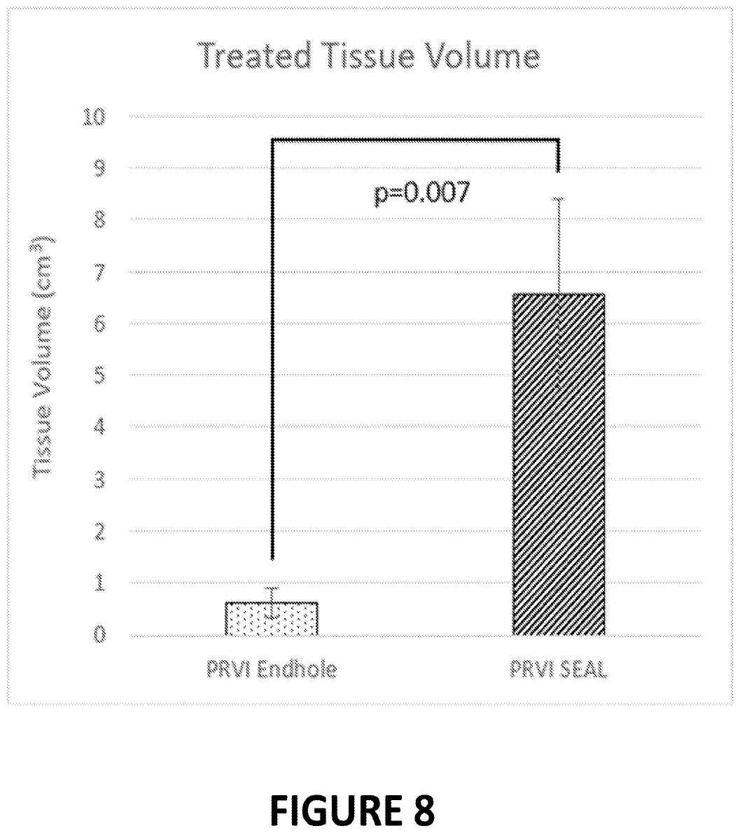

FIG. 8 depicts the treated volume for the SEAL Device as compared to the end hole catheter with the outlier data removed. In this regard, using the SEAL Device resulted in a 10.6 fold increase in treated tissue volume.

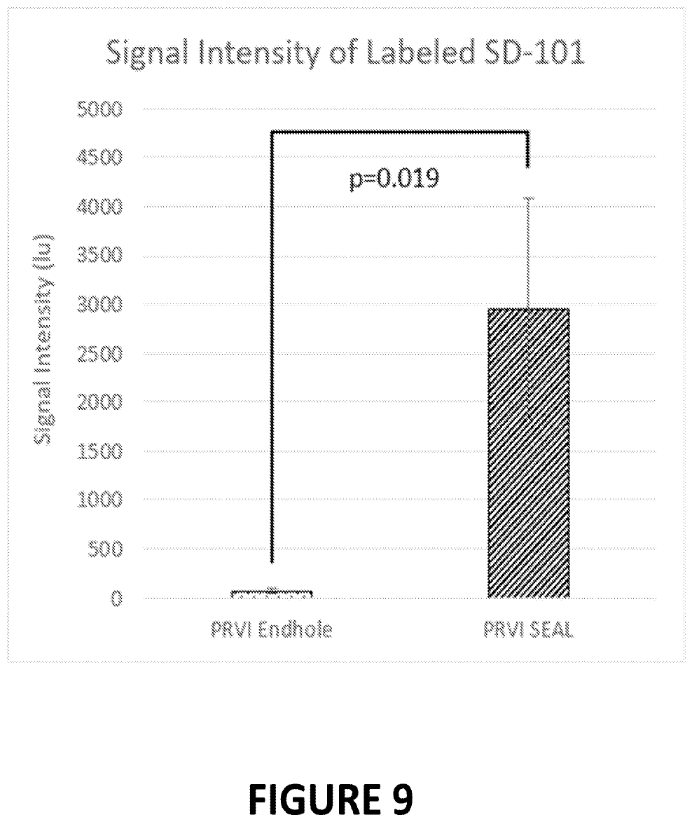

FIG. 9 depicts the treated signal intensity for the SEAL Device as compared to the end hole catheter with the outlier data removed. In this regard, using the SEAL Device resulted in a 46 fold increase in labeled SD-101 delivered to the tissue as measured by signal intensity.

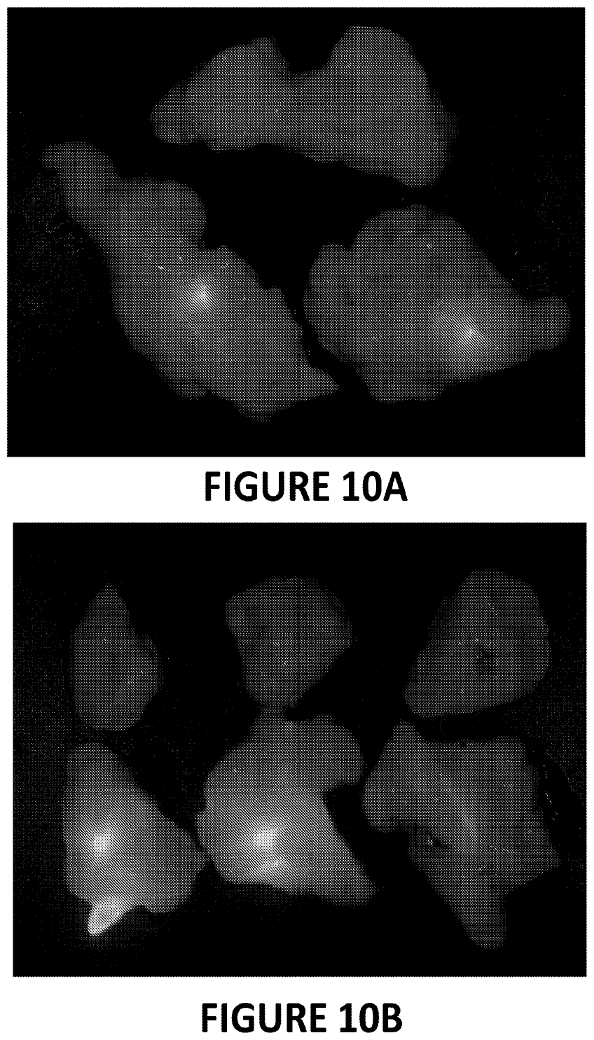

FIG. 10A depicts the distribution pattern of labeled SD-101 delivered by the end hole catheter, while FIG. 10B depicts the distribution pattern of labeled SD-101 delivered by the SEAL device. In this regard, infusion with the end hole catheter led to deposition of labeled SD-101 along the vein with minimal penetration into the tissue. However, therapy delivery using the SEAL Device resulted in penetration into the tissue outside of the primary draining vein.

### Example 4

In the present example, it was hypothesized that pancreatic retrograde venous infusions (PRVI) using a PEDD, e.g., the SEAL Device, of a TLR9 agonist, SD-101, can enhance response rates to CPI in patients with locally advanced PDAC. Furthermore, through distal effects of SD-101 PEDD/PRVI in patients also receiving CPI systemic infusions, extra-pancreatic lesions may also benefit from enhanced immune-responsiveness. As such, responsiveness of locally advanced PDAC to immunotherapy can be optimized while enabling systemic anti-tumor immunity. Accordingly, through more effective delivery of SD-101 to PDAC tumors and elimination of suppressive immune cell such as MDSC, higher CPI responsiveness may be possible in patients with locally advanced PDAC.

The combinatorial approach can be conducted in two phases, i.e., Phases 1 and 1b. In this regard, the primary objective for Phase 1 is to determine the maximum tolerated dose (MTD) of SD 101 alone via PEDD/PRVI. Further, the secondary objective is to assess the Response Evaluation Criteria in Solid Tumors (RECIST) v1.1 overall response rate (ORR). With regard to the Phase 1b, the primary objective is to determine the safety of SD-101 via PEDD/PRVI in combination with pembrolizumab and to assess the Response Evaluation Criteria in Solid Tumors (RECIST) v1.1 overall response rate (ORR) and 12-month progression-free survival (PFS) (co-primary endpoints). Further, the secondary objective is to assess the 12-month overall survival (OS) and progression-free survival to PEDD/PRVI of SD-101 in combination with intravenous (IV) immunological checkpoint blockade. Further, another secondary objective is to assess preliminary efficacy in terms of RECIST for immune based therapeutics (iRECIST) ORR, RECIST 1.1 pancreatic-specific response rate (PRR), duration of response (DOR), and clinical benefit (complete response [CR]+partial response [PR]+stable disease [SD]).

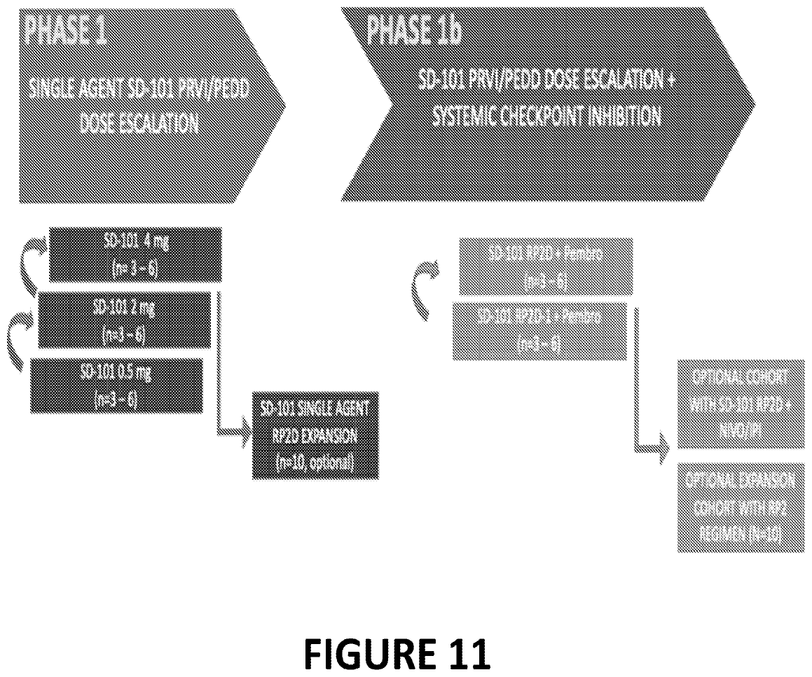

The overall design for the study can be found in FIG. 11.

In Phase 1, escalating doses of SD-101 will be administered alone via PEDD/PRVI into the regional vessels the pancreas containing the locally advanced tumor. Following determination of the recommended MTD or optimal dose of SD-101 for PEDD/PRVI, the study can progress to Phase 1b to assess safety of concomitant SD-101 and CPI usage, along with preliminary efficacy. Patients in Phase 1b can receive the SD-101 dose selected from Phase 1 in the presence of systemic anti-PD-1 checkpoint blockade. SD-101 can be administered over 2 cycles, with 1 dose per cycle and each cycle being one month apart.

Following the SD-101 infusions for each patient in phase 1, an overnight in-hospital observation or admission can be required. If the safety of SD-101 PEDD/PRVI is established in Phase 1, overnight observation in Phase 1b is at the discretion of the treating physician for the subsequent SD-101 infusion. If infusions are performed on an outpatient basis, the patient can be observed for a minimum of 6 hours post infusion before being discharged, if clinically stable. If there are any >Grade 2 events related to SD-101 PEDD/PRVI that required in-patient therapy following the first infusion, the patient can be kept for overnight observation or admission following each subsequent SD-101 infusion.

### Inclusion Criteria

According to an embodiment, to be included in the study, the patient must meet all of the following criteria for inclusion:
1. Patients ≥18 years of age with histologically or cytologically confirmed evaluable or measurable locally advanced unresectable PDAC according to RECIST v1.1 criteria. Imaging confirmation centrally of unresectable disease as defined by NCCN is required.
2. Performance status score of 0 or 1 on the Eastern Cooperative Oncology Group (ECOG) scale (scores range from 0 to 5, with higher numbers reflecting greater disability)
3. Suitable venous anatomy on CT venogram as defined by absence of portal, splenic, or superior mesenteric vein complete occlusion.
4. Having received standard of care chemoradiation therapy or a systemic chemotherapy regimen without a complete radiographic response. Standard of care chemotherapy include gemcitabine+nab-paclitaxel, or FOLFIRINOX; for others discuss with medical monitor. Radiation with or without concurrent chemotherapy is also acceptable as a standard of care regimen.
5. Adequate hematologic and organ function.
6. Able to understand the study and provide written informed consent prior to any study procedures
7. Has not received prior cytotoxic chemotherapy, targeted therapy, or external radiation therapy within 14 days prior to screening
8. Has no prior history of or other concurrent malignancy unless the malignancy is clinically insignificant, no ongoing treatment is required, and the patient is clinically stable
9. Has measurable disease in the liver according to RECIST v.1.1 criteria
10. Has a life expectancy of >3 months at screening as estimated by the investigator
11. Has a QTc interval ≤480 msec
12. All associated clinically significant (in the judgment of the investigator) drug-related toxicity from previous cancer therapy must be resolved (to Grade ≤1 or the patient's pretreatment level) prior to study treatment administration (Grade 2 alopecia and endocrinopathies controlled on replacement therapy are allowed).
13. Has adequate organ function at screening as evidence by:
   Platelet count >100,000/μL
   Hemoglobin ≥8.0 g/dL
   White blood cell count (WBC) >2,000/L
   Serum creatinine ≤2.0 mg/dL unless the measured creatinine clearance is ≥30 mL/min calculated by Cockcroft-Gault formula.
   Total and direct bilirubin ≤2.0× the upper limit of normal (ULN) and alkaline phosphatase ≤5×ULN. For patients with documented Gilbert's disease, total bilirubin up to 3.0 mg/dL is allowed.
   ALT and AST ≤5×ULN
   Amylase and lipase ≤3×ULN
   Prothrombin time/International Normalized Ratio (INR) or activated partial thromboplastin time (aPTT) test results at screening ≤1.5×ULN (this applies only to patients who do not receive therapeutic anticoagulation; patients receiving therapeutic anticoagulation should be on a stable dose for at least 4 weeks prior to the first dose of study intervention)
14. Females of childbearing potential must be nonpregnant and nonlactating, or post-menopausal, and have a negative serum human chorionic gonadotropin (hCG) pregnancy test result at screening and prior to the first dose of study intervention.
   Females of childbearing potential must agree to abstain from sexual activity with nonsterilized male partners, or if sexually active with a nonsterilized male partner must agree to use highly effective methods of contraception from screening, throughout the study and agree to continue using such precautions for 100 days after the final dose of study intervention.
   Nonsterilized males who are sexually active with a female of childbearing potential must agree to use effective methods of contraception and avoid sperm donation from Day 1 throughout the study and for 30 days after the final dose of study intervention.

### Phase 1

Dose-escalation cohort of PEDD/PRVI of SD 101 monotherapy (2 cycles, 1 month apart) using a standard 3+3 design:
Dose level 1: 0.5 mg (n=3-6)
Dose level 2: 2 mg (n=3-6)
Dose level 3: 4 mg (n=3-6)
Dose level 4*: 8 mg (n=3-6)
Dose level 5* 12 mg (n=3-6)

*Dose levels 4 and 5 are optional. If the PEDD/PRVI procedures and dose levels 1-3 were well tolerated, but clinical and/or immunologic activity were minimal, the additional dose levels may be enrolled. Clinical activity will be defined as more than one RECIST 1.1 CR or PR across dose levels, at least two patients with a >20% decrease in SUV level on FDG-PET scan, or at least two patients with >20% decrease in serum CA-19-9 levels. Minimal immunologic responsiveness will be defined as the absence of a decrease in intra-tumoral MDSC, increase in intra-tumoral CD8+ T cells, or increase in IFNα/IFNγ related gene signatures.

Enrollment of the first 2 patients at each dose level can be staggered by at least 72 hours. Progression to higher dose levels can be delayed 7 days following the final infusion in the last subject at the preceding dose level. Progression to the next dosing cohort can occur following review of safety data and confirmation by the SRC. An optional expansion group of 10 patients at the SD-101 monotherapy MTD or optimal dose may proceed concurrently with Phase 1b.

### Phase 1b

Standard 3+3 design dose re-escalation of PEDD/PRVI of SD-101 (2 cycles, 1 dose per cycle with one month in between cycles) together with intravenous (IV) pembrolizumab 200 mg every 3 weeks (Q3W) to identify the MTD or optimal dose of SD-101 via PEDD/PRVI with systemic CPI:
Dose level 1: Pembrolizumab together with PEDD/PRVI of SD-101 at 1 dose level below the MTD or optimal dose from Phase 1 (i.e., MTD-1 or optimal dose-1) (n=3-6)
Dose level 2: Pembrolizumab together with PEDD/PRVI of SD-101 at the MTD or optimal dose from Phase 1 (n=3-6), or if MTD-1 or optimal dose −1 not tolerated, will de-escalate to MTD-2 or optimal dose-2

Enrollment of the first 2 patients at each dose level can be staggered by at least 48 hours. Progression to higher dose levels can be delayed 7 days following the final infusion in the last subject at the preceding dose level. Progression to the next dosing cohort can occur following review of safety data and confirmation by the SRC. An optional expansion group of 10-20 patients at the MTD or optimal dose may proceed.

There will be optional expansion cohorts at the RP2D of SD-101 in combination with pembrolizumab or with the RP2D of SD-101 in combination with nivolumab+ipilimumab.

### Study Interventions

TABLE 17

| Intervention | | Keytruda ® | Opdivo ® | Yervoy ® |
|---|---|---|---|---|
| Name | SD-101 | (pembrolizumab)^{a} | (nivolumab) ^{a} | (ipilimumab) ^{a} |
| Type | Drug (ODN) | Biologic (antibody) | Biologic (antibody) | Biologic |
| | | | | (antibody) |
| Dose | Solution | Solution for | Solution for | Solution for |
| Formulation | | injection | injection | injection |
| Unit Dose | 13.4 mg/mL* | 100 mg/4 mL | 40 mg/4 mL | 5 mg/mL |
| Strength(s) | | | 100 mg/10 mL | |
| | | | 240 mg/24 mL | |
| Dosage Level(s) | 0.5, 2, or 4 mg | 200 mg | 1 mg/kg IV | 3 mg/kg IV |
| | | | 240 mg IV | |
| Route of | Intravascular | IV | IV | IV |
| Administration | injection by | | | |
| | PEDD/PRVI | | | |
| | over 30-60 | | | |
| | minutes | | | |
| Use | Experimental | Background | Background | Background |
| | | intervention | intervention | intervention |
| IMP and NIMP | IMP | NIMP | NIMP | NIMP |
| Sourcing | TriSalus | Commercial - site | Commercial - site | Commercial -site |
| | | supply | supply | supply |
| Packaging and | Single-use vial | Single-use vial | Single-dose vials | Single-dose vials |
| Labeling | | | | |

*Unit dose strength of SD-101 reflects only SD-101 oligo.
Abbreviations: IMP = investigational medicinal product; IV = intravenous; NIMP = non-investigational medicinal product.

### SD-101 Administration

SD-101 can be administered using the SEAL Device. The SEAL Device is a 5.0F to 3.1F tapered coaxial infusion catheter having a 0.021″ inner lumen with an expandable valve at the distal end that serves as the conduit for physician-specified agents. The valve is designed to variably expand within vessels ranging from 2 to 6 mm in diameter and forms a fluid impermeable barrier in the presence of retrograde flow. The device is further adapted to interface with standard invasive blood pressure (IBP) transducers in a manner that allows for continuous pressure monitoring in vasculature distal to the valve throughout infusion of therapeutic. During infusion, the device blocks all retrograde flow and generates pressure in the vessel, resulting in the perfusion of the venous and capillary network isolated by the device.

### CPI Administration

Pembrolizumab, nivolumab, and ipilimumab can be administered as separate IV infusions at the dose levels specified in Table 12.

### Duration of SD-101 Administration (all Participants in Phase 1 and Phase 1b)

Up to 2 doses (over 2 cycles with a month in between cycles). The second cycle of SD-101 may be omitted on the basis of toxicity or tolerability. All patients receiving at least 1 PEDD/PRVI dose of SD-101 will be considered evaluable.

### Duration of CPI Administration

Up to 6 months of pembrolizumab 200 mg Q3W.

### PEDD/PRVI of SD-101

The SD 101 solution can be infused via the pancreatic venous system using the SEAL Device. In brief, the procedure involves performance or a transhepatic or transjugular venogram to define target draining pancreatic veins that would allow selective drug delivery to the region of the gland containing the tumor. In some cases, one vein may be sufficient, while in others, drug delivery via 2 or more branches may be required. Off-target branches may require embolic occlusion.
SD-101 Therapeutic Volume: 10 mL
SD-101 Therapeutic Doses: 0.5 mg, 2 mg, 4 mg
SD-101 Diluent: Commercially available, preservative free, 0.9% Sodium Chloride, USP (sterile isotonic saline).

### Tumor Response Evaluations

All patients can undergo imaging with magnetic resonance imaging (MRI) or computed tomography (CT) to assess extent and metabolic activity of disease in the pancreas, as well as assess any extra-pancreatic lesions, pancreas biopsies and assays of CTC, circulating cytokines, and other immunologic correlatives. Tumor response can be measured radiographically using standard RECIST v1.1 criteria. Official response scoring (per RECIST v1.1) can be preliminarily assessed 21 days following each infusion. Final response scoring can be determined 42 days following the second infusion to ensure that pseudo-progression is ruled out and that initial response is confirmed. Imaging procedures can occur every 90 days thereafter. Local imaging reads can be utilized for response assessment during Phase 1. Independent central review for response assessment may be performed during Phase 1b.

Up to 2 PDAC tumor core needle biopsy sessions will take place by endoscopic ultrasound (EUS):
A baseline biopsy will be obtained on one week before the first infusion of SD-101
A second biopsy will be performed one week after the second cycle of SD-101 (before SD-101 Infusion #2).
During each biopsy session, 3 core needle samples will be taken from the PDAC tumor under EUS guidance.
Pathologic response will be assessed based on review by the site pathologist with scoring of necrosis and fibrosis within tumor samples. If multiple sites are participating, pathology review will be centralized to a single site.
Immunologic correlative studies will be performed per protocol Intra-vascular pressure recordings will be obtained during each infusion session.

### Pharmacokinetics

Blood samples can be collected to characterize SD-101 systemic exposure after PEDD/PRVI. No sampling or testing will be done for CPI concentrations. Tumor levels of SD-101 can be measured in the post-infusion biopsy specimens.

### Pharmacodynamics

Blood samples can be collected for the measurement of CTC, circulating cytokines, and other immunologic correlatives including interferon alpha (IFN-α) and interferon gamma (IFN-γ) related gene signatures, which may be more informative than pharmacokinetic (PK) assessments for this class of therapeutic.

### Safety

For Phase 1 and 1b, an SRC composed of the study investigators can be utilized to ensure patient safety, decide on dose cohort transitions, decide whether to continue or terminate the study early, as well as oversee the validity and integrity of the study conduct and data. Membership may be changed based on the phase of the study. During the Phase 1b portion of the study, a statistician may be included.

Safety assessments include adverse events (AEs), clinical laboratory testing, vital signs, physical examinations, and electrocardiograms (ECGs) as clinically indicated.

The following are considered DLTs when observed during either SD-101 cycle or within 2 weeks after the last SD-101 dose in Cycle 2 and are considered attributable to study intervention (SD-101 or CPI therapy) and/or the PEDD device:
≥Grade 3 cytokine release syndrome (CRS) per National Cancer Institute (NCI) Common Terminology Criteria for Adverse Events (CTCAE)
Autoimmune AE ≥Grade 3 per NCI CTCAE
Allergic reaction AE ≥Grade 3 per NCI CTCAE
Grade 4 hematologic AE per NCI CTCAE that does not recover to ≤Grade 2 within 7 days
Grade 4 AE per NCI CTCAE in any organ system including pancreatitis

Patients who develop a DLT during either cycle of SD 101 can be permanently discontinued from study interventions unless adequate justification is provided that an alternative approach (e.g., with dose modification) is expected to be reasonably safe given the specific DLT. The patient can be treated according to clinical practice and monitored for resolution of the toxicity.

SD-101 and/or CPI therapy can be permanently discontinued for severe or life-threatening infusion-related reactions. Dose interruptions, delays, or discontinuation of SD-101 and/or CPI therapy is required when a patient has a Grade 3 or higher immune-mediated reaction. Discontinuation of SD-101 and/or CPI therapy is required when a patient meets one of the conditions outlined below:
Patient has clinical or radiographic evidence of severe pancreatitis
Patient has clinical evidence of portal hypertension including but not limited to moderate or severe ascites, that is clinically significant, or variceal bleeding.

In some embodiments, the present invention relates to the use of a TLR9 agonist in the manufacture of a medicament for treating a solid tumor in the pancreas, said method comprising administering the TLR9 agonist to a patient in need thereof, wherein the TLR9 agonist is administered through a device by PRVI to such solid tumor in the pancreas.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method for treating pancreatic cancer comprising administering to a subject in need thereof a therapeutically effective amount of a toll-like receptor 9 agonist having the structure: 5′-TCG AAC GTT CGA ACG TTC GAA CGT TCG AAT-3′ (SEQ ID NO: 1).

2. The of claim 1, wherein the TLR9 agonist is administered through a device by pancreatic retrograde venous infusion (PRVI).

3. The method of claim 1, wherein the TLR9 agonist is administered is selected from the group consisting of 0.5 mg, 2 mg, 4 mg, 8 mg, or 12 mg.

4. The method of claim 2, wherein the TLR9 agonist may be administered through a catheter device.

5. The method of claim 4, wherein the catheter device is a temporary occlusion device.

6. The method of claim 2, wherein the TLR9 agonist is administered through the catheter device via pressure-enabled drug delivery.

7. The method of claim 7, wherein the TLR9 agonist is administered with a rate of infusion of about 1 mL per minute to about 10 ml per minute.

8. The method of claim 7, wherein the TLR9 agonist is administered for a period of time of two to twenty minutes.

9. The method of claim 1, wherein the TLR9 agonist is administered in combination with one or more checkpoint inhibitors, wherein the checkpoint inhibitors are administered systemically, either concurrently, before, or after the administration of the TLR9 agonist.

10. The method of claim 10, wherein the one or more checkpoint inhibitors include at least one of nivolumab, pembrolizumab, and cemiplimab, atezolizumab, avelumab, and durvalumab, and ipilimumab.

11. The method of claim 1, wherein the administration of the TLR9 agonist comprises a dosing regimen comprising cycles, in which one or more of the cycles comprise the administration of the TLR9 agonist via a catheter device by pancreatic retrograde venous infusion (PRVI) followed by the systemic administration of a checkpoint inhibitor.

12. The method of claim 10, wherein the one or more checkpoint inhibitors include at least one of nivolumab, pembrolizumab, and cemiplimab, atezolizumab, avelumab, and durvalumab, and ipilimumab.


SEQUENCE LISTING (1 sequence)

SEQ ID NO: 1 - DNA, 30 bp, Artificial Sequence
  Description of Artificial Sequence Synthetic oligonucleotide
tcgaacgttcgaacgttcgaacgttcgaat